(19) United States
(12) Patent Application Publication
(10) Pub. No.: US 2024/0041869 A1
(43) Pub. Date: Feb. 8, 2024

(54) OPHTHALMIC COMPOSITION COMPRISING AN ANTI-ALLERGEN AND A REDNESS REDUCTION AGENT

(71) Applicant: Bausch + Lomb Ireland Limited, Dublin (IE)

(72) Inventors: Zora Marlowe, Rochester, NY (US); Martin Coffey, Pittsford, NY (US)

(73) Assignee: Bausch + Lomb Ireland Limited, Dublin (IE)

(21) Appl. No.: 18/365,019

(22) Filed: Aug. 3, 2023

Related U.S. Application Data
(60) Provisional application No. 63/370,409, filed on Aug. 4, 2022.

(51) Int. Cl. A61K 31/498; A61K 31/451; A61K 31/335; A61K 47/10; A61K 47/26; A61K 47/32; A61K 47/18

(52) U.S. Cl. CPC A61K 31/498; A61K 31/451; A61K 31/335; A61K 47/10; A61K 47/26; A61K 47/32; A61K 47/186

(57) ABSTRACT

Ophthalmic compositions comprising an anti-allergen and a redness reduction agent, such as brimonidine or a pharmaceutically acceptable salt thereof, for treating itching and redness in a single composition. The ophthalmic compositions may be provided in a container closure system, such as a low density polyethylene bottle. Container closure systems and kits comprising the ophthalmic compositions are disclosed.

This application claims benefit of priority from U.S. Provisional Application No. 63/370,409, filed Aug. 4, 2022, which is incorporated herein by reference in its entirety.

Disclosed herein are ophthalmic compositions comprising an anti-allergen and a redness reduction agent, such as brimonidine or a pharmaceutically acceptable salt thereof. The ophthalmic compositions provide itch relief and redness relief in a single composition. The ophthalmic compositions are stable for both active ingredients, provide efficient drug delivery, are well tolerated by the eye, and have good preservative efficacy with low concentrations of benzalkonium chloride. The ophthalmic compositions may be provided in a container closure system, such as a low density polyethylene (LDPE) bottle.

Ophthalmic compositions are useful for the treatment and temporary prevention of the signs and symptoms of ocular conditions, including conjunctivitis, allergic conjunctivitis, itching of the eye, dry eye, inflammatory dry eye, and redness of the eye. Methods of treating ocular conditions include administering to a human subject suffering therefrom or susceptible thereto an ophthalmic composition.

Ophthalmic compositions may be formulated as single or multi dose units, with or without the use of a preservative, and may be manufactured by mixing various ingredients. The compositions may be packaged in single or multiple dosage forms, such as vials, ampoules, bottles, tubes, syringes, dispenser packages, or other suitable containers made from materials such as glass or plastic. In some cases, the packaging for the ophthalmic composition may be free or substantially free of antioxidant (e.g., as used in compositions described in U.S. Pat. Nos. 6,455,547 and 6,576,649).

Typically, ophthalmic compositions are administered as eye drops, with one or more drops of the composition being applied to an eye of the subject suffering from or susceptible to ocular conditions one or more times per day, although the frequency of administration of such compositions may be dependent on multiple factors, including the makeup of the particular composition and the condition for which the compositions are used.

Ophthalmic solutions may contain buffers, various surfactants, stabilizers, isotonic agents and the like which aid in making the ophthalmic compositions more comfortable to the user. Oftentimes the ophthalmic solutions contain such agents and the like to maintain chemical stability and a predictable level of efficacy over a predetermined or expected lifetime.

Ophthalmic compositions typically have a pH anywhere from 4 to 8. The pH value is generally targeted to provide a specific level or range, which provides the least amount of discomfort to the end user. Conventionally, a buffer (e.g., buffers including citrates, phosphates, borates, bicarbonates, sodium salts, potassium salts, etc. or a buffer with intrinsic antimicrobial properties such as a sodium borate/boric acid buffer) is used to achieve and maintain a desired pH of the compositions, and/or an acid or base is added to adjust the pH of the compositions to the desired level. In particular, a desired pH for a particular formulation is typically determined to provide combined attributes of acceptable chemical stability of the formulation and comfort to the end user.

Of particular importance for efficacy and commercialization of ophthalmic solutions is solution stability. Maintenance of efficacy and stability of ophthalmic solutions may be required to meet various federal health and safety regulations, e.g., shelf life testing, sterility, etc. For example, ophthalmic solutions may be required to contain expiration dates posted on their container, which may be predicated on the stability of the active ingredients and other conditions inherent in the formulation and environmental exposures of the product.

Solution stability may be dependent on the interactions of all compounds present in the formulation as well as temperature and pH.

Oftentimes stabilizing agents, although effective in maintaining specific properties of the formulation, are undesirable ingredients as they may cause adverse side effects in end-users or promote the degradation of active agents in the formulation.

Furthermore, it may be desirable for an ophthalmic composition to include a plurality of active agents. In such situations, it may be difficult or uneconomical to meet a particular shelf life target or regulatory requirements due to some instability of the combination of the active agents or other interaction with components of a formulation. This may be the result of some chemical reactivity or incompatibility of the compounds or salts thereof, for example, which leads to degradation of one or more of the active agents. Such degradation shortens the shelf life of the solution and may render the formulation pharmaceutically ineffective or non-compliant with federal regulatory requirements.

The present disclosure provides ophthalmic compositions comprising (a) an anti-allergen and (b) a redness reduction agent. In some embodiments, the ophthalmic composition comprises (a) an anti-allergen and (b) brimonidine and/or a pharmaceutically acceptable salt thereof at a concentration from about 0.010% (w/v) to about 0.050% (w/v). In some embodiments, the ophthalmic composition comprises (a) ketotifen and/or a pharmaceutically acceptable salt thereof at a concentration from about 0.010% (w/v) to about 0.050% (w/v) and (b) brimonidine and/or a pharmaceutically acceptable salt thereof at a concentration from about 0.010% (w/v) to about 0.050% (w/v). In some embodiments, the ophthalmic composition comprises (a) olopatadine and/or a pharmaceutically acceptable salt thereof at a concentration from about 0.100% (w/v) to about 0.250% (w/v) and (b) brimonidine and/or a pharmaceutically acceptable salt thereof at a concentration from about 0.010% (w/v) to about 0.050% (w/v).

In some embodiments, the ophthalmic composition further comprises (c) two or more tonicity agents, (d) povidone, (e) an optional preservative, and (f) water. In some embodiments, the ophthalmic composition further comprises (c) two or more non-ionic tonicity agents in an amount such that the composition has an osmolality of about 275 mOsm/kg to about 385 mOsm/kg, (d) povidone at a concentration from about 0.15% (w/v) to about 0.45% (w/v), (e) an optional preservative, and (f) water.

The present disclosure further provides ophthalmic compositions comprising (a) an anti-allergen and (b) a redness reduction agent, wherein the composition is in a container closure system. In some embodiments, the ophthalmic composition comprises (a) an anti-allergen and (b) brimonidine and/or a pharmaceutically acceptable salt thereof at a concentration from about (w/v) to about 0.050% (w/v), wherein the composition is in a container closure system. In some embodiments, the ophthalmic composition comprises (a) ketotifen and/or a pharmaceutically acceptable salt thereof at a concentration from about 0.010% (w/v) to about 0.050% (w/v) and (b) brimonidine and/or a pharmaceutically acceptable salt thereof at a concentration from about (w/v) to about 0.050% (w/v), wherein the composition is in a container closure system. In some embodiments, the ophthalmic composition comprises (a) olopatadine and/or a pharmaceutically acceptable salt thereof at a concentration from about 0.100% (w/v) to about (w/v) and (b) brimonidine and/or a pharmaceutically acceptable salt thereof at a concentration from about 0.010% (w/v) to about 0.050% (w/v), wherein the composition is in a container closure system.

In some embodiments, the ophthalmic composition further comprises (c) two or more tonicity agents, (d) povidone, (e) an optional preservative, and (f) water, wherein the composition is in an LDPE container closure system. In some embodiments, the ophthalmic composition further comprises (c) two or more non-ionic tonicity agents in an amount such that the composition has an osmolality of about 275 mOsm/kg to about 385 mOsm/kg, (d) povidone at a concentration from about 0.15% (w/v) to about 0.45% (w/v), (e) an optional preservative, and (f) water, wherein the composition is in an LDPE container closure system.

The present disclosure also provides a container closure system containing an ophthalmic composition comprising (a) an anti-allergen and (b) a redness reduction agent. In some embodiments, the container closure system contains an ophthalmic composition comprising (a) an anti-allergen and (b) brimonidine and/or a pharmaceutically acceptable salt thereof at a concentration from about 0.010% (w/v) to about 0.050% (w/v). In some embodiments, the container closure system contains an ophthalmic composition comprising (a) ketotifen and/or a pharmaceutically acceptable salt thereof at a concentration from about 0.010% (w/v) to about (w/v) and (b) brimonidine and/or a pharmaceutically acceptable salt thereof at a concentration from about 0.010% (w/v) to about 0.050% (w/v). In some embodiments, the container closure system contains an ophthalmic composition comprising (a) olopatadine and/or a pharmaceutically acceptable salt thereof at a concentration from about 0.100% (w/v) to about (w/v) and (b) brimonidine and/or a pharmaceutically acceptable salt thereof at a concentration from about 0.010% (w/v) to about 0.050% (w/v).

In some embodiments, the container closure system contains an ophthalmic composition further comprising (c) two or more tonicity agents, (d) povidone, (e) an optional preservative, and (f) water, wherein the container closure system is in an LDPE container closure system. In some embodiments, the container closure system contains an ophthalmic composition further comprising (c) two or more non-ionic tonicity agents in an amount such that the composition has an osmolality of about 275 mOsm/kg to about 385 mOsm/kg, (d) povidone at a concentration from about 0.15% (w/v) to about 0.45% (w/v), (e) an optional preservative, and (f) water, wherein the container closure system is in an LDPE container closure system.

The present disclosure further provides kits comprising a container closure system containing an ophthalmic composition comprising (a) an anti-allergen and (b) a redness reduction agent. In some embodiments, the kit comprising a container closure system contains an ophthalmic composition comprising (a) an anti-allergen and (b) brimonidine and/or a pharmaceutically acceptable salt thereof at a concentration from about 0.010% (w/v) to about 0.050% (w/v). In some embodiments, the kit comprising a container closure system contains an ophthalmic composition comprising (a) ketotifen and/or a pharmaceutically acceptable salt thereof at a concentration from about 0.010% (w/v) to about 0.050% (w/v) and (b) brimonidine and/or a pharmaceutically acceptable salt thereof at a concentration from about 0.010% (w/v) to about 0.050% (w/v). In some embodiments, the kit comprising a container closure system contains an ophthalmic composition comprising (a) olopatadine and/or a pharmaceutically acceptable salt thereof at a concentration from about 0.100% (w/v) to about 0.250% (w/v) and (b) brimonidine and/or a pharmaceutically acceptable salt thereof at a concentration from about 0.010% (w/v) to about 0.050% (w/v).

In some embodiments, the kit comprising a container closure system contains an ophthalmic composition further comprising (c) two or more tonicity agents, (d) povidone, (e) an optional preservative, and (f) water, wherein the container closure system is in an LDPE container closure system. In some embodiments, the kit comprising a container closure system contains an ophthalmic composition further comprising (c) two or more non-ionic tonicity agents in an amount such that the composition has an osmolality of about 275 mOsm/kg to about 385 mOsm/kg, (d) povidone at a concentration from about 0.15% (w/v) to about 0.45% (w/v), (e) an optional preservative, and (f) water, wherein the container closure system is in an LDPE container closure system.

These and other features, aspects, and advantages of the disclosure will be apparent from a reading of the following detailed, which are briefly described below. Other aspect and advantages of the disclosed subject matter will become apparent from the following.

Definitions of certain terms as used in this application are provided below. Unless defined otherwise, all technical and scientific terms used herein have the normal and common meaning that would be commonly understood by one of ordinary skill in the art to which this disclosure belongs.

As used herein, “a” or “an” entity refers to one or more of that entity, e.g., “a compound” refers to one or more compounds or at least one compound unless stated otherwise. As such, the terms “a” (or “an”), “one or more”, and “at least one” are used interchangeably herein.

As used herein, the term “about” means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 5%. Numeric values modified by the term “about” include the specific identified value. For example, “about 100” means a number ranging from 95 to 105, including 95, 100, and 105. Recitation of ranges of values herein are merely intended to serve as a shorthand method of referring individually to each separate value falling within the range, unless otherwise indicated herein, and each separate value is incorporated into the specification as if it were individually recited herein.

As used herein, “administration” of a compound or composition to a patient refers to any route of introducing or delivering the compound or composition to a subject. Administration includes self-administration and the administration by another.

As used herein, a “condition,” “disorder,” or “disease” relates to any unhealthy or abnormal state.

As used herein, an “effective amount” or “effective dose” refers to an amount of a compound or composition that treats, upon single or multiple dose administration, a patient suffering from a disorder, disease, or condition. An effective amount can be determined by the attending diagnostician through the use of known techniques and by observing results obtained under analogous circumstances. In determining the effective amount, a number of factors are considered by the attending diagnostician, including, but not limited to: the species of patient; its size, age, and general health; the specific condition, disorder, or disease involved; the degree of or involvement or the severity of the condition, disorder, or disease, the response of the individual patient; the particular compound administered; the mode of administration; the bioavailability characteristics of the preparation administered; the dose regimen selected; the use of concomitant medication; and other relevant circumstances.

As used herein, the term “in combination with,” when referring to two or more compounds, agents, or additional active pharmaceutical ingredients, means the administration of two or more compounds, agents, or active pharmaceutical ingredients to the patient prior to, concurrent with, or subsequent to each other during a treatment period. Unless specified otherwise, the two or more compounds, agents, or active pharmaceutical ingredients may be administered on different schedules during the treatment period, such as, e.g., with one or more compounds, agents, or active pharmaceutical ingredients being administered once a day and one or more other compounds, agents, or active pharmaceutical ingredients being administered twice a day.

The terms “patient,” “subject,” “individual,” and the like, as used herein, are interchangeable and refer to any animal, which may be a human or a non-human animal.

As used herein, “prevention” of or “preventing” a disorder, disease, or condition refers to reduction of or reducing the occurrence of the disorder, disease, or condition in a treated sample relative to an untreated control sample, and includes delaying onset, progression, or reduction of severity of one or more symptoms of the disorder or condition relative to the untreated control sample.

As used herein, the term “pharmaceutically acceptable salt” refers to a salt form of a compound wherein the salt is nontoxic and include such salts derived from suitable inorganic and organic acids and bases. Pharmaceutically acceptable salts are well known in the art. For example, S. M. Berge, et al. describes pharmaceutically acceptable salts in detail in J. Pharmaceutical Sciences, 1977, 66, 1-19. Non-limiting examples of suitable pharmaceutically acceptable acid addition salts include chlorides, bromides, sulfates, nitrates, phosphates, sulfonates, methane sulfonates, formates, tartrates, maleates, succinates, malonates, citrates, benzoates, salicylates, and ascorbates. Non-limiting examples of suitable pharmaceutically acceptable base addition salts include sodium, potassium, lithium, ammonium (substituted and unsubstituted), calcium, magnesium, iron, zinc, copper, manganese, and aluminum salts. Non-limiting examples of pharmaceutically acceptable salts include pharmaceutically acceptable salts derived from appropriate bases include alkali metal, alkaline earth metal, ammonium, and N⁺(C₁₋₄alkyl)₄ salts. This disclosure also envisions the quaternization of any basic nitrogen-containing groups of the compounds disclosed herein. Pharmaceutically acceptable salts may, for example, be obtained using standard procedures well known in the field of pharmaceuticals. One of ordinary skill in the art will recognize that the stability and other properties of different pharmaceutically acceptable salts of the recited components herein may differ and will consider these differences when selecting suitable pharmaceutically acceptable salt(s).

As used herein, a “pharmaceutically acceptable excipient” refers to a carrier or an excipient that is useful in preparing a pharmaceutical composition. For example, a pharmaceutically acceptable excipient is generally safe and includes carriers and excipients that are generally considered acceptable for mammalian pharmaceutical use. As a non-limiting example, pharmaceutically acceptable excipients may be solid, semi-solid, or liquid materials which, in the aggregate, can serve as a vehicle or medium for active ingredients. Some examples of pharmaceutically acceptable excipients are found in Remington's Pharmaceutical Sciences and the Handbook of Pharmaceutical Excipients and include diluents, vehicles, carriers, ointment bases, binders, disintegrates, lubricants, glidants, sweetening agents, flavoring agents, gel bases, sustained release matrices, stabilizing agents, preservatives, solvents, suspending agents, buffers, emulsifiers, dyes, propellants, coating agents, and others.

As used herein, the term “reduce” refers to altering negatively by at least 5% including, but not limited to, altering negatively by 5%, altering negatively by 10%, altering negatively by 25%, altering negatively by 30%, altering negatively by 50%, altering negatively by 75%, or altering negatively by 100%.

As used herein, the term “treat,” “treating,” or “treatment,” when used in connection with a disorder or condition, includes any effect, e.g., lessening, reducing, modulating, ameliorating, or eliminating, that results in the improvement of the disorder or condition. Improvements in or lessening the severity of any symptom of the disorder or condition can be readily assessed according to standard methods and techniques known in the art.

As will be understood by one of ordinary skill in the art, each range disclosed herein includes all possible subranges as well as individual numerical values within that range, including endpoints. As a non-limiting example, a range of “0.001% to 0.02%” includes and would be understood to specifically disclose subranges such as “0.004% to 0.01%,” “0.005% to 0.02%,” etc., as well as all individual numbers within the disclosed range, for example, 0.001%, 0.004%, 0.01%, 0.02%, etc.

Claims or descriptions that include “or” or “and/or” between members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all the group members are present in, employed in, or otherwise relevant to a given product or process.

The ophthalmic compositions of the present disclosure include one or more anti-allergens. In some embodiments, the composition comprises two or more anti-allergens. As used herein, the term “anti-allergen” refers to compounds and compositions that relieve and/or control allergic conjunctivitis. As used herein, “anti-allergen” includes antihistamines and mast cell stabilizers. In some embodiments, the anti-allergen is a dual acting antihistamine/mast cell stabilizer. In some embodiments, the anti-allergen may be chosen from acrivastine, azelastine, bepotastine besilate, cetirizine, chlorpheniramine, cromolyn sodium, desloratadine, ebastine, emedastine, epinastine, fexofenadine, ketotifen, levocabastine, levocetirizine, lodoxamide, loratadine, mizolastine, nedocrimil, norastemizole, olopatadine, combinations thereof, analogs thereof, as well as pharmaceutically acceptable salts thereof (e.g., ketotifen fumarate or olopatadine HCl).

In some embodiments of the ophthalmic compositions of the present disclosure, the anti-allergen may be present at a concentration from about 0.0001% (w/v) to about 0.500% (w/v). In other embodiments of the compositions, the anti-allergen may be present at a concentration from about 0.0001% (w/v) to about 0.050% (w/v). In some embodiments of the compositions, the anti-allergen may be present at a concentration from about 0.001% (w/v) to about 0.050% (w/v). In some embodiments of the compositions, the anti-allergen may be present at a concentration from about 0.010% (w/v) to about 0.050% (w/v), such as from 0.010% (w/v) to 0.050% (w/v). In some embodiments of the compositions, the anti-allergen may be present at a concentration from about 0.020% (w/v) to about 0.040% (w/v), such as from 0.020% (w/v) to 0.040% (w/v).

In some embodiments of the ophthalmic compositions of the present disclosure, the anti-allergen is ketotifen and/or a pharmaceutically acceptable salt thereof (such as ketotifen fumarate), which may be present at concentrations from about 0.0001% (w/v) to about 0.500% (w/v). In some embodiments, ketotifen and/or a pharmaceutically acceptable salt thereof may be present at a concentration from about 0.0001% (w/v) to about 0.050% (w/v). In some embodiments, ketotifen and/or a pharmaceutically acceptable salt thereof may be present at a concentration from about 0.001% (w/v) to about 0.050% (w/v), such as from 0.001% (w/v) to (w/v). In some embodiments, ketotifen and/or a pharmaceutically acceptable salt thereof may be present at a concentration from about 0.010% (w/v) to about 0.050% (w/v), such as from (w/v) to 0.050% (w/v).

In some embodiments, ketotifen and/or a pharmaceutically acceptable salt thereof (such as ketotifen fumarate) may be present at a concentration from about 0.020% (w/v) to about 0.040% (w/v), such as from 0.020% (w/v) to 0.040% (w/v). In some embodiments, ketotifen and/or a pharmaceutically acceptable salt thereof may be present at a concentration of about 0.020% (w/v), about 0.025% (w/v), about 0.030% (w/v), about 0.035% (w/v), or about 0.040% (w/v).

In other embodiments of the compositions, the anti-allergen may be present at a concentration from about 0.001% (w/v) to about 0.500% (w/v). In some embodiments of the compositions, the anti-allergen may be present at a concentration from about 0.010% (w/v) to about 0.500% (w/v), such as from 0.010% (w/v) to 0.500% (w/v). In some embodiments of the compositions, the anti-allergen may be present at a concentration from about 0.100% (w/v) to about 0.500% (w/v), such as from 0.100% (w/v) to 0.500% (w/v).

In some embodiments of the ophthalmic compositions of the present disclosure, the anti-allergen is olopatadine and/or a pharmaceutically acceptable salt thereof (such as olopatadine HCl), which may be present at concentrations from about 0.0001% (w/v) to about 0.500% (w/v). In some embodiments, olopatadine and/or a pharmaceutically acceptable salt thereof may be present at a concentration from about 0.001% (w/v) to about 0.500% (w/v). In some embodiments, olopatadine and/or a pharmaceutically acceptable salt thereof may be present at a concentration from about 0.010% (w/v) to about 0.500% (w/v), such as from 0.010% (w/v) to 0.500% (w/v). In some embodiments, olopatadine and/or a pharmaceutically acceptable salt thereof may be present at a concentration from about 0.100% (w/v) to about 0.500% (w/v), such as from 0.100% (w/v) to (w/v).

In some embodiments, olopatadine and/or a pharmaceutically acceptable salt thereof (such as olopatadine HCl), may be present at a concentration from about 0.100% (w/v) to about (w/v), such as from 0.100% (w/v) to 0.250% (w/v). In some embodiments, olopatadine and/or a pharmaceutically acceptable salt thereof may be present at a concentration from about (w/v) to about 0.150% (w/v), such as from 0.100% (w/v) to 0.150% (w/v). In some embodiments, olopatadine and/or a pharmaceutically acceptable salt thereof may be present at a concentration of about 0.110% (w/v), about 0.111% (w/v), about 0.112% (w/v), about 0.113% (w/v), about 0.114% (w/v), or about 0.115% (w/v).

The ophthalmic compositions of the present disclosure include one or more redness reduction agents. In some embodiments, the composition comprises two or more redness reduction agents. In some embodiments, the redness reduction agent is an adrenergic receptor agonist. In some embodiments, the redness reduction agent is an α-1 adrenergic receptor agonist. In some embodiments, the redness reduction agent is an α-2 adrenergic receptor agonist. In some embodiments, the redness reduction agent is a selective α-2 adrenergic receptor agonist, which has a binding activity of 100-fold or greater for α-2 over α-1 adrenergic receptors. A selective α-2 adrenergic receptor agonist may be chosen from apraclonidine, mivazerol, clonidine, brimonidine, alpha methyl dopa, guanfacine, dexemeditomidine, (+)-(S)-4-[1-(2,3-dimethyl-phenyl)-ethyl]-1,3-dihydro-imidazole-2-thione, 1-[(imidazolidin-2-yl)imino]indazole, combinations thereof, analogs thereof, as well as pharmaceutically acceptable salts thereof (e.g., brimonidine tartrate). As long as a particular isomer, salt, analog, prodrug or other derivative of a selective α-2 adrenergic receptor agonist functions as a selective α-2 agonist, it may be used for the purposes of the present disclosure.

Other redness reduction agents that may be used, include, but are not limited to, phentolamine, naphazoline, tetrahydrozoline, methoxamine, ephedrine, phenylephrine, other vasoconstrictors, combinations thereof, as well as pharmaceutically acceptable salts thereof (e.g., naphazoline hydrochloride and tetrahydrozoline hydrochloride).

In some embodiments of the ophthalmic compositions of the present disclosure, the redness reduction agents may be present at a concentration from about 0.0001% (w/v) to about (w/v). In some embodiments of the compositions, the redness reduction agents may be present at a concentration from about 0.001% (w/v) to about 0.050% (w/v). In some embodiments of the compositions, the redness reduction agents may be present at a concentration from about (w/v) to about 0.050% (w/v), such as from 0.010% (w/v) to 0.050% (w/v). In some embodiments of the compositions, the redness reduction agents may be present at a concentration from about 0.001% (w/v) to about 0.025% (w/v), such as from 0.001% (w/v) to 0.025% (w/v). In some embodiments of the compositions, the redness reduction agents may be present at a concentration from about 0.010% (w/v) to about 0.025% (w/v), such as from 0.010% (w/v) to (w/v).

It is preferred that a concentration of a redness reduction agent is below its vasoconstriction vs. concentration plateau. Typically, the optimal concentration is 10% to 90% above the minimal threshold of measurable vasoconstriction for a particular agent, or below that of the plateau maximum concentration, and is preferably within the about 25% to about 75% range of either of these benchmarks. The term “plateau maximum concentration” means the concentration above which there is no or minimal further vasoconstriction effect. Other considerations in choosing an agent are blood brain permeability and any possible side effects and other systemic reactions.

In some embodiments of the ophthalmic compositions of the present disclosure, the redness reduction agent is brimonidine and/or a pharmaceutically acceptable salt thereof, which may be present at concentrations from about 0.001% (w/v) to about 0.050% (w/v). In some embodiments, brimonidine and/or a pharmaceutically acceptable salt thereof may be present at a concentration from about 0.010% (w/v) to about 0.050% (w/v). In some embodiments, brimonidine and/or a pharmaceutically acceptable salt thereof may be present at a concentration from about 0.015% (w/v) to about 0.045% (w/v), such as from 0.015% (w/v) to 0.045% (w/v). In some embodiments, brimonidine and/or a pharmaceutically acceptable salt thereof may be present at a concentration from about 0.015% (w/v) to about 0.035% (w/v), such as from 0.015% (w/v) to 0.035% (w/v). In some embodiments, brimonidine and/or a pharmaceutically acceptable salt thereof may be present at a concentration from about 0.020% (w/v) to about 0.030% (w/v), such as from 0.020% (w/v) to 0.030% (w/v). In some embodiments, brimonidine and/or a pharmaceutically acceptable salt thereof may be present at a concentration of about 0.020% (w/v), about 0.025% (w/v), about 0.030% (w/v), such as from 0.025% (w/v) to 0.030% (w/v).

The ophthalmic compositions of the present disclosure may also include additional non-therapeutic components, which include, but are not limited to, preservatives, delivery vehicles, tonicity agents, buffers, pH adjustors, antioxidants, and water.

The ophthalmic compositions of the present disclosure are adjusted with a tonicity agent. In some embodiments, the ophthalmic composition is adjusted with a tonicity agent to a given osmolality. In some embodiments, the tonicity agent is a nonionic tonicity agent chosen from glycerol, urea, sorbitol, mannitol, propylene glycol, dextrose, and other ophthalmically acceptable nonionic tonicity agents. In some embodiments, the ophthalmic compositions of the present disclosure comprise glycerol, propylene glycol, mannitol, sorbitol, or combinations thereof. In other embodiments, the nonionic tonicity agent is used in combination with an ionic salt tonicity agent, such as sodium chloride, at sufficient concentrations to provide a given osmolality.

In some embodiments, the ophthalmic compositions of the present disclosure are adjusted with two or more tonicity agents, such as two or more non-ionic tonicity agents. In certain embodiments, the compositions comprise glycerol and mannitol. In certain embodiments, the compositions comprise glycerol and propylene glycol. In certain embodiments, the compositions comprise glycerol and sorbitol. In certain embodiments, the compositions comprise mannitol and propylene glycol. In certain embodiments, the ophthalmic compositions of the present disclosure comprise glycerol, propylene glycol, and mannitol.

When present in the ophthalmic compositions of the present disclosure, each tonicity agent may be used in a concentration of about 0.1% (w/v) to about 10.0% (w/v). In some embodiments, the compositions have a tonicity agent in concentration of about 0.5% (w/v) to about 3.5% (w/v), such as 0.5% (w/v), 1.0% (w/v), 1.5% (w/v), 2.0% (w/v), 2.5% (w/v), 3.0% (w/v), or 3.5% (w/v). In some embodiments, the composition has glycerol in a concentration of about 0.5% (w/v) to about 6.0% (w/v), such as from about 1.0% (w/v) to about 2.0% (w/v), including 1.0% (w/v), 1.1% (w/v), 1.2% (w/v), 1.3% (w/v), 1.4% (w/v), 1.5% (w/v), 1.6% (w/v), 1.7% (w/v), 1.8% (w/v), 1.9% (w/v), and 2.0% (w/v) In some embodiments, the composition has mannitol in a concentration of about 0.5% (w/v) to about 2.3% (w/v), such as from about 1.0% (w/v) to about 2.0% (w/v), including 2.0% (w/v). In some embodiments, the composition has propylene glycol in a concentration of about 0.5% (w/v) to about 1.5% (w/v), including 1.0% (w/v). In some embodiments, the composition has sorbitol in a concentration of about 1.0% (w/v) to about 2.5% (w/v), including 2.0% (w/v).

In some embodiments, the ophthalmic compositions of the present disclosure are isotonic. In some embodiments, the compositions are adjusted to approximate the osmotic pressure of normal lachrymal fluids, which, as stated in U.S. Pat. No. 6,274,626, is equivalent to a 0.9% solution of sodium chloride or a 2.5% solution of glycerol. Osmotic pressure, measured as osmolality, is generally about 225 mOsm/kg to about 400 mOsm/kg for conventional ophthalmic compositions.

In some embodiments, the ophthalmic compositions of the present disclosure have an osmolality of about 225 mOsm/kg to about 400 mOsm/kg, such as 225 mOsm/kg to 400 mOsm/kg. In some embodiments, the compositions have an osmolality of about 275 mOsm/kg to about 385 mOsm/kg, such as of 275 mOsm/kg to 385 mOsm/kg. In some embodiments, the compositions have an osmolality of about 285 mOsm/kg to about 355 mOsm/kg, such as 285 mOsm/kg to 355 mOsm/kg. In some embodiments, the compositions have an osmolality of about 275 mOsm/kg to about 325 mOsm/kg, such as about 275, about 280, about 285, about 290, about 295, about 300, about 305, about 315, about 320, or about 325 mOsm/kg. In some embodiments, the composition has an osmolality of about 290 mOsm/kg. In some embodiments, the composition has an osmolality of about 295 mOsm/kg. In some embodiments, the composition has an osmolality of about 300 mOsm/kg.

However, in further embodiments, the ophthalmic compositions of the present disclosure may be formulated to osmolality in the range from about 400 to about 875 mOsm/kg, for some desired purposes. In particular, such osmolality may be employed if the composition is formulated to be further well tolerated by a user. For example, US Patent Appl. Publ. No. 2006/0148899, incorporated herein by reference in its entirety, provides for ophthalmic compositions having an osmolality from 400 to 875 mOsm/kg, which have been found to provide comfort to a user.

In some embodiments, the ophthalmic composition of the present disclosure includes one or more preservatives. For example, a preservative may be used when the composition is packaged for multidose units but may be absent from the composition in single dose units of the composition. The ophthalmic compositions of the present disclosure exhibit preservative efficacy according to USP/EP criteria, including when borate free. In some embodiments, the ophthalmic compositions of the present disclosure exhibit preservative efficacy according to USP/EP criteria at low benzalkonium chloride concentrations, including at 0 ppm BAK, when borate free.

When a preservative is to be present, any preservative for ophthalmic compositions may be used, including, but are not limited to, benzalkonium chloride, chlorobutanol, thimerosal, phenylmercuric acetate, benzethonium chloride, cetrimide, stabilized oxychloro complex, chlorite, or phenylmercuric nitrate. However, the ophthalmic compositions of the present disclosure are borate free. When a preservative is used in the composition, the preservative is typically provided in a concentration of about 0.0005% (w/v) to about 10.0% (w/v), although other concentrations may be used.

In other embodiments, the ophthalmic composition of the present disclosure are preservative free. Such preservative free compositions can be packaged either in a single-dose unit container closure system or inside a preservative free multidose container closure system. In some embodiments, the preservative free ophthalmic compositions of the present disclosure are packed in a single-dose unit container closure system intended for single-use by the patient. In other embodiments, the preservative free ophthalmic compositions of the present disclosure are packed in preservative free multidose container closure systems that enable the composition to be kept sterile/germ-free even after multiple uses by the patient.

In some embodiments, the ophthalmic composition of the present disclosure comprises benzalkonium chloride in an effective amount as a preservative. In some embodiments, the composition comprises from about 0.0005% (w/v) to about 10.0% (w/v) benzalkonium chloride. In some embodiments, the composition comprises from about 0.002% (w/v) to about 1.0% (w/v) benzalkonium chloride. In some embodiments, the composition comprises from about 0.002% (w/v) to about 0.50% (w/v) benzalkonium chloride, such as from 0.002% (w/v) to about 0.50% (w/v). In some embodiments, the composition comprises from about 0.002% (w/v) to about (w/v) benzalkonium chloride, such as from 0.002% (w/v) to about 0.050% (w/v). In some embodiments, the composition comprises from about 0.002% (w/v) to about 0.004% (w/v) benzalkonium chloride, such as from 0.002% (w/v) to about 0.004% (w/v).

In some embodiments, the composition comprises about 0.0005% (w/v) benzalkonium chloride. In some embodiments, the composition comprises about 0.001% (w/v) benzalkonium chloride. In some embodiments, the composition comprises about 0.0015% (w/v) benzalkonium chloride. In some embodiments, the composition comprises about 0.002% (w/v) benzalkonium chloride. In some embodiments, the composition comprises about 0.0025% (w/v) benzalkonium chloride. In some embodiments, the composition comprises about 0.003% (w/v) benzalkonium chloride. In some embodiments, the composition comprises about 0.0035% (w/v) benzalkonium chloride. In some embodiments, the composition comprises about 0.004% (w/v) benzalkonium chloride.

In some embodiments, the ophthalmic composition of the present disclosure has no benzalkonium chloride.

In some embodiments, the ophthalmic composition of the present disclosure may include one or more acids or bases to adjust the pH of the composition and/or may include a buffer to achieve (and maintain) the desired pH of the compositions.

In some embodiments, the ophthalmic composition of the present disclosure includes one or more pH adjustors. It is understood that acids or bases can be used to adjust the pH of the composition as needed. When a pH adjustor is to be present, any pH adjustor for ophthalmic compositions may be used, including, but are not limited to hydrochloric acid, sodium hydroxide, and tromethamine. Typically, only small amounts of an acid or base will be needed to adjust the pH of the composition, such as 0.05% to 0.1% of a 1N solution.

The pH of the composition is adjusted for optimization of stability and patient tolerability. In some embodiments, the ophthalmic composition of the present disclosure is adjusted to a pH between about 4.4 to about 6.0, although the compositions may also have a pH outside of this range. In some embodiments, the composition has a pH between about 5.2 to about such as between 5.2 to 5.8. In some embodiments, the composition has a pH between about to about 5.75. In some embodiments, the composition has a pH of 4.4, 4.5, 4.6, 4.7, 4.8, 4.8, 5.1, 5.2, 5.3, 5.4, 5.5, 5.6, 5.7, 5.8, 5.9, or 6.0.

In some embodiments, the ophthalmic composition of the present disclosure includes one or more buffers. When a buffer is to be present, any buffer for ophthalmic compositions may be used, including, but are not limited to, acetate buffers, bicarbonate buffers, citrate buffers, and phosphate buffers, potassium salt buffers, sodium salt buffers. However, the ophthalmic compositions of the present disclosure are borate free. If present, a buffer is used in the ophthalmic composition at a concentration above its “break point concentration,” which is defined generally as the concentration of a buffer that is insufficient to maintain the pH of a solution comprising one or more active agents at a temperature for a given time duration. The buffer is typically provided in a concentration from about 1 millimolar (“mM”) to about 10 mM, such as from about 2 mM to about 6 mM and from about 3 mM to about 4 mM. It has been observed that in some embodiments, buffers decrease the stability of the active ingredients (such as ketotifen). Accordingly, in some embodiments, the ophthalmic composition of the present disclosure is buffer free.

In some embodiments, the ophthalmic composition of the present disclosure includes one or more antioxidants. In some embodiments, the ophthalmic composition of the present disclosure does not include antioxidants. When an antioxidant is to be present, any antioxidant for ophthalmic compositions may be used, including, but are not limited to, sodium metabisulfite, sodium thiosulfate, acetylcysteine, butylated hydroxyanisole and butylated hydroxytoluene.

In some embodiments, the ophthalmic composition of the present disclosure includes one or more delivery vehicles. Delivery vehicles include, but are not limited to, polyvinyl alcohol, povidone, hydroxypropyl methyl cellulose, poloxamers, carboxymethyl cellulose, hydroxyethyl cellulose, and water (purified, distilled, deionized). It is also possible to use a physiological saline solution as a major vehicle.

In some embodiments, the ophthalmic composition of the present disclosure further comprises an effective amount of an additional therapeutic agent, such as an additional active ingredient.

In some embodiments, the additional therapeutic agent is chosen from antihistamines. In some embodiments, the additional therapeutic agent is chosen from pheniramine maleate, carbinoxamine maleate, S-(+)-chlorpheniramine maleate, diphenhydramine, hydroxyzine, and azelastine. In some embodiments, the additional therapeutic agent is pheniramine maleate. In some embodiments, the additional therapeutic agent is chosen from antihistamines, anti-inflammatory agents, and antibiotics. In some embodiments, the additional therapeutic agent is chosen from anti-inflammatory agents and antibiotics. In some embodiments, the additional therapeutic agent is chosen from anti-inflammatory agents. In some embodiments, the additional therapeutic agent is chosen from antibiotics. In some embodiments, the additional therapeutic agent is chosen from fluoroquinolones. In some embodiments, the additional therapeutic agent is besifloxacin.

In some embodiments, the ophthalmic composition of the present disclosure further comprises a pharmaceutically acceptable excipient chosen from humectants, suspending agents, viscosity-increasing agents, wetting agents, solubilizing agents, chelating agents, stabilizers, and nitrous oxide inhibitors.

In some embodiments, the suspending agents and viscosity-increasing agents are chosen from povidone K90, USP/EP, HPMC E15LV (USP), polycarbophil, HPMC E4M USP, and combinations thereof.

In some embodiments, the wetting agents and solubilizing agents are chosen from povidone, poloxamer 407, PS80, and combinations thereof. In some embodiments, the ophthalmic composition of the present disclosure includes povidone. When present in the ophthalmic compositions of the present disclosure, povidone may additionally act as a lubricant, protecting against irritation and dryness.

When present in the ophthalmic composition as a wetting agent, povidone is used at a concentration from about 0.010% (w/v) to about 1.20% (w/v). In some embodiments, povidone may be present at a concentration from about 0.050% (w/v) to about 1.00% (w/v). In some embodiments, povidone may be present at a concentration from about 0.075% (w/v) to about 0.75% (w/v), such as from 0.075% (w/v) to about 0.75% (w/v). In some embodiments, povidone may be present at a concentration from about 0.15% (w/v) to about 0.45% (w/v), such as from (w/v) to about 0.45% (w/v). In some embodiments, povidone may be present at a concentration of about 0.15% (w/v), about 0.20% (w/v), about 0.25% (w/v), about 0.30% (w/v), about 0.35% (w/v), about 0.40% (w/v), or about 0.45% (w/v). In some embodiments, the ophthalmic compositions of the present disclosure, which comprise povidone, do not cause a stinging sensation and are comfortable to the eye when applied. Povidone has been observed to improve the stability of the anti-allergen (such as ketotifen fumarate) of the ophthalmic compositions of the present disclosure under high temperature stress conditions.

In some embodiments, the chelating agent is chosen from disodium EDTA, dihydrate USP/EP, and combinations thereof.

In some embodiments, the stabilizers are chosen from ascorbic acid, citric acid, sodium benzoate, calcium propionate, sodium erythorbate, sodium nitrite, calcium sorbate, potassium sorbate, BHA, BHT, EDTA, tocopherols, and combinations thereof. In other embodiments, the stabilizer contains an anionic component, such as peroxide class preservative. The stabilizer allows one to achieve greater penetration of lipophilic membranes. However, in some embodiments, the ophthalmic composition of the present disclosure does not include an antioxidant.

In some embodiments, the nitrous oxide inhibitors are chosen from L-NAME (L-NG-Nitroarginine methyl ester), L-NIL (N-6-(1-iminoethyl)-L-lysine dihydrochloride), L-NIO (N-5-(1-iminoethyl)-L-ornithine dihydrochloride), L-canavine, and combinations thereof.

In some embodiments, the ophthalmic composition comprises (a) an anti-allergen and (b) brimonidine and/or a pharmaceutically acceptable salt thereof.

In some embodiments, the ophthalmic composition comprises (a) an anti-allergen, (b) brimonidine and/or a pharmaceutically acceptable salt thereof at a concentration from about (w/v) to about 0.050% (w/v), (c) two or more non-ionic tonicity agents in an amount such that the composition has an osmolality of about 275 mOsm/kg to about 385 mOsm/kg, (d) povidone at a concentration from about 0.15% (w/v) to about 0.45% (w/v), (e) an optional preservative, and (f) water.

In some embodiments, the ophthalmic composition comprises (a) ketotifen or a pharmaceutically acceptable salt thereof and (b) brimonidine or a pharmaceutically acceptable salt thereof.

In some embodiments, the ophthalmic composition comprises (a) ketotifen and/or a pharmaceutically acceptable salt thereof at a concentration from about 0.010% (w/v) to about (w/v), (b) brimonidine and/or a pharmaceutically acceptable salt thereof at a concentration from about 0.010% (w/v) to about 0.050% (w/v), (c) two or more non-ionic tonicity agents in an amount such that the composition has an osmolality of about 275 mOsm/kg to about 385 mOsm/kg, (d) povidone at a concentration from about 0.15% (w/v) to about 0.45% (w/v), (e) an optional preservative, and (f) water.

In some embodiments, the ophthalmic composition comprises (a) ketotifen fumarate and (b) brimonidine tartrate.

In some embodiments, the ophthalmic composition comprises (a) ketotifen fumarate at a concentration from about 0.010% (w/v) to about 0.050% (w/v), (b) brimonidine tartrate from about 0.010% (w/v) to about 0.050% (w/v), (c) two or more non-ionic tonicity agents in an amount such that the composition has an osmolality of about 275 mOsm/kg to about 385 mOsm/kg, (d) povidone at a concentration from about 0.15% (w/v) to about 0.45% (w/v), (e) an optional preservative, and (f) water.

In some embodiments, the ophthalmic composition comprises (a) olopatadine or a pharmaceutically acceptable salt thereof and (b) brimonidine or a pharmaceutically acceptable salt thereof.

In some embodiments, the ophthalmic composition comprises (a) olopatadine and/or a pharmaceutically acceptable salt thereof at a concentration from about 0.100% (w/v) to about (w/v), (b) brimonidine and/or a pharmaceutically acceptable salt thereof at a concentration from about 0.010% (w/v) to about 0.050% (w/v), (c) two or more non-ionic tonicity agents in an amount such that the composition has an osmolality of about 275 mOsm/kg to about 385 mOsm/kg, (d) povidone at a concentration from about 0.15% (w/v) to about 0.45% (w/v), (e) an optional preservative, and (f) water.

In some embodiments, the ophthalmic composition comprises (a) olopatadine HCl and (b) brimonidine tartrate.

In some embodiments, the ophthalmic composition comprises (a) olopatadine HCl at a concentration from about 0.100% (w/v) to about 0.250% (w/v), (b) brimonidine tartrate from about (w/v) to about 0.050% (w/v), (c) two or more non-ionic tonicity agents in an amount such that the composition has an osmolality of about 275 mOsm/kg to about 385 mOsm/kg, (d) povidone at a concentration from about 0.15% (w/v) to about 0.45% (w/v), (e) an optional preservative, and (f) water.

In some embodiments, the stability of the active ingredients of the ophthalmic composition of the present disclosure is maintained at an acceptable level at 25° C. and 40% relative humidity for at least two year after the manufacture of such composition. In some embodiments, the stability of the active ingredients of the ophthalmic composition of the present disclosure is maintained at an acceptable level at 40° C. and 20% relative humidity for at least two year after the manufacture of such composition. In further embodiments, such an extended period of time is at least three years or longer. Stability of the active ingredient(s) of the ophthalmic composition is observed when less than about 20% (or alternatively, less than 15%, or less than 10%, or less than 5% or less than 2% or less than 1%) (by weight) of each active ingredient has degraded or changed in such period of time.

In some embodiments, after the ophthalmic composition has been maintained at 25° C. and 40% relative humidity for at least two years, less than 20% of the active ingredient, such as ketotifen or a pharmaceutically acceptable salt thereof and brimonidine or a pharmaceutically acceptable salt thereof, originally in the ophthalmic composition has degraded or changed. In some embodiments, less than 10%, such as less than 5%, less than 4%, less than 3%, less than 2%, less than 1%, or less than 0.5% of each active ingredient, such as ketotifen or a pharmaceutically acceptable salt thereof and brimonidine or a pharmaceutically acceptable salt thereof, originally in the ophthalmic composition has degraded or changed.

In some embodiments, after the ophthalmic composition has been maintained at 40° C. and 25% relative humidity for at least two years, less than 20% of the active ingredient, such as ketotifen or a pharmaceutically acceptable salt thereof and brimonidine or a pharmaceutically acceptable salt thereof, originally in the ophthalmic composition has degraded or changed. In some embodiments, less than 10%, such as less than 5%, less than 4%, less than 3%, less than 2%, less than 1%, or less than 0.5% of each active ingredient, such as ketotifen or a pharmaceutically acceptable salt thereof and brimonidine or a pharmaceutically acceptable salt thereof, originally in the ophthalmic composition has degraded or changed.

The impurities resulting from degradation of an active ingredient, such as ketotifen or a pharmaceutically acceptable salt thereof and brimonidine or a pharmaceutically acceptable salt thereof, are characterized based on industry standards relative to the initial amount of that active ingredient in the ophthalmic composition. In some embodiments after at least two years, degradation products of an active ingredient constitute no more than about 5.0% of the initial amount of the active ingredient. In some embodiments, no more than about 4.0%, no more than about 3.0%, no more than about 2.0%, no more than about 1.0%, or no more than 0.5%.

Ophthalmic compositions are typically kept in container closure systems (e.g., vials, ampoules, bottles, tubes, syringes, dispenser packages, or other suitable containers). In some embodiments, the compositions may be packaged either in a single-dose unit container closure system or multi-dose container closure system. Container closure systems may be composed of one or more materials appropriate for its use, such as aluminum, glass, polypropylene, polyethylene (e.g., low-density polyethylene and high-density polyethylene), polyethylene terephthalate, and polyethylene terephthalate glycol. Plastic container closure systems weigh less, are more resistant to shock and other mechanical influences, cost less, and offer more design possibilities than glass. Polyethylene, e.g., low-density polyethylene (LDPE), without or with additives, and polypropylene are the plastics required by the European Pharmacopoeia. In one embodiment of the present disclosure, container closure systems may be squeezable to allow delivery of the ophthalmic composition as drops to the eye.

Polypropylene is known to be stronger, stiffer, and more high temperature-resistant than low-density polyethylene. However, polypropylene has a poorer resistance to oxidation agents such as oxygen and acids, which can lead to fissures and yellowing of the plastic. Also polypropylene does not provide superior flexibility and processability as compared to polyethylene. Polypropylene is not a first choice for container closure systems for sterile compositions, especially for blow fill seal technology. Also polypropylene is not a cost effective option as compared to polyethylene.

In one embodiment, a single container closure system can comprise one or more compartments for containing a provided ophthalmic composition, and/or appropriate aqueous carrier for suspension or dilution. In some embodiments, a single container can be appropriate for modification such that the container can receive a physical modification so as to allow combination of compartments and/or components of individual compartments. For example, a foil or plastic bag can comprise two or more compartments separated by a perforated seal which can be broken so as to allow combination of contents of two individual compartments once the signal to break the seal is generated.

Ophthalmic compositions disclosed herein may be prepared according to any known method for the manufacture of ophthalmic formulations or preparations. As will be appreciated by those of ordinary skill in the art, a number of methods are known. In some embodiments, the ophthalmic compositions disclosed herein may be prepared by any conventional technique, such as, e.g., those described in Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988-1999, Marcel Dekker, New York.

In some embodiments, the ophthalmic compositions of the present disclosure are in LDPE container closure systems (e.g., vials, ampoules, bottles, tubes, syringes, dispenser packages, or other suitable containers). Exemplary LDPE container closure systems include white LDPE bottles and natural LDPE bottles. LDPE containers are squeezable but also are semi-permeable to volatile compounds. Whereas ophthalmic compositions with ketotifen fumarate had been observed to have dramatic losses during stability testing, the ophthalmic composition of the present disclosure is stable in LDPE container closure systems (e.g., white and natural LDPE bottles). It is desirable for an ophthalmic composition to demonstrate stability in multiple types of container closure systems, each of a different material type.

The ophthalmic compositions of the present disclosure in LDPE container closure systems include one or more anti-allergens. In some embodiments, the composition comprises two or more anti-allergens. As used herein, the term “anti-allergen” refers to compounds and compositions that relieve and/or control allergic conjunctivitis. As used herein, “anti-allergen” includes antihistamines and mast cell stabilizers. In some embodiments, the anti-allergen is a dual acting antihistamine/mast cell stabilizer. In some embodiments, the anti-allergen may be chosen from acrivastine, azelastine, bepotastine besilate, cetirizine, chlorpheniramine, cromolyn sodium, desloratadine, ebastine, emedastine, epinastine, fexofenadine, ketotifen, levocabastine, levocetirizine, lodoxamide, loratadine, mizolastine, nedocrimil, norastemizole, olopatadine, combinations thereof, analogs thereof, as well as pharmaceutically acceptable salts thereof (e.g., ketotifen fumarate or olopatadine HCl).

In some embodiments of the ophthalmic compositions of the present disclosure in LDPE container closure systems, the anti-allergen may be present at a concentration from about (w/v) to about 0.500% (w/v). In other embodiments of the compositions, the anti-allergen may be present at a concentration from about 0.0001% (w/v) to about 0.050% (w/v). In some embodiments of the compositions, the anti-allergen may be present at a concentration from about (w/v) to about 0.050% (w/v). In some embodiments of the compositions, the anti-allergen may be present at a concentration from about 0.010% (w/v) to about 0.050% (w/v), such as from (w/v) to 0.050% (w/v). In some embodiments of the compositions, the anti-allergen may be present at a concentration from about 0.020% (w/v) to about 0.040% (w/v), such as from (w/v) to 0.040% (w/v).

In some embodiments of the ophthalmic compositions of the present disclosure in LDPE container closure systems, the anti-allergen is ketotifen and/or a pharmaceutically acceptable salt thereof (such as ketotifen fumarate), which may be present at concentrations from about 0.0001% (w/v) to about 0.500% (w/v). In some embodiments, ketotifen and/or a pharmaceutically acceptable salt thereof may be present at a concentration from about 0.0001% (w/v) to about 0.050% (w/v). In some embodiments, ketotifen and/or a pharmaceutically acceptable salt thereof may be present at a concentration from about 0.001% (w/v) to about 0.050% (w/v), such as from 0.001% (w/v) to 0.050% (w/v). In some embodiments, ketotifen and/or a pharmaceutically acceptable salt thereof may be present at a concentration from about 0.010% (w/v) to about 0.050% (w/v), such as from 0.010% (w/v) to 0.050% (w/v).

In some embodiments in LDPE container closure systems, ketotifen and/or a pharmaceutically acceptable salt thereof (such as ketotifen fumarate) may be present at a concentration from about 0.020% (w/v) to about 0.040% (w/v), such as from 0.020% (w/v) to (w/v). In some embodiments, ketotifen and/or a pharmaceutically acceptable salt thereof may be present at a concentration of about 0.020% (w/v), about 0.025% (w/v), about 0.030% (w/v), about 0.035% (w/v), or about 0.040% (w/v).

In other embodiments of the compositions in LDPE container closure systems, the anti-allergen may be present at a concentration from about 0.001% (w/v) to about 0.500% (w/v). In some embodiments of the compositions, the anti-allergen may be present at a concentration from about 0.010% (w/v) to about 0.500% (w/v), such as from 0.010% (w/v) to 0.500% (w/v). In some embodiments of the compositions, the anti-allergen may be present at a concentration from about (w/v) to about 0.500% (w/v), such as from 0.100% (w/v) to 0.500% (w/v).

In some embodiments of the ophthalmic compositions of the present disclosure in LDPE container closure systems, the anti-allergen is olopatadine and/or a pharmaceutically acceptable salt thereof (such as olopatadine HCl), which may be present at concentrations from about 0.0001% (w/v) to about 0.500% (w/v). In some embodiments, olopatadine and/or a pharmaceutically acceptable salt thereof may be present at a concentration from about 0.001% (w/v) to about 0.500% (w/v). In some embodiments, olopatadine and/or a pharmaceutically acceptable salt thereof may be present at a concentration from about 0.010% (w/v) to about 0.500% (w/v), such as from 0.010% (w/v) to 0.500% (w/v). In some embodiments, olopatadine and/or a pharmaceutically acceptable salt thereof may be present at a concentration from about 0.100% (w/v) to about 0.500% (w/v), such as from 0.100% (w/v) to 0.500% (w/v).

In some embodiments in LDPE container closure systems, olopatadine and/or a pharmaceutically acceptable salt thereof (such as olopatadine HCl), may be present at a concentration from about 0.100% (w/v) to about 0.250% (w/v), such as from 0.100% (w/v) to (w/v). In some embodiments, olopatadine and/or a pharmaceutically acceptable salt thereof may be present at a concentration from about 0.100% (w/v) to about 0.150% (w/v), such as from 0.100% (w/v) to 0.150% (w/v). In some embodiments, olopatadine and/or a pharmaceutically acceptable salt thereof may be present at a concentration of about 0.110% (w/v), about 0.111% (w/v), about 0.112% (w/v), about 0.113% (w/v), about 0.114% (w/v), or about (w/v).

The ophthalmic compositions of the present disclosure in LDPE container closure systems include one or more redness reduction agents. In some embodiments, the composition comprises two or more redness reduction agents. In some embodiments, the redness reduction agent is an adrenergic receptor agonist. In some embodiments, the redness reduction agent is an α-1 adrenergic receptor agonist. In some embodiments, the redness reduction agent is an α-2 adrenergic receptor agonist. In some embodiments, the redness reduction agent is a selective α-2 adrenergic receptor agonist, which has a binding activity of 100-fold or greater for α-2 over α-1 adrenergic receptors. A selective α-2 adrenergic receptor agonist may be chosen from apraclonidine, mivazerol, clonidine, brimonidine, alpha methyl dopa, guanfacine, dexemeditomidine, (+)-(S)-4-[1-(2,3-dimethyl-phenyl)-ethyl]-1,3-dihydro-imidazole-2-thione, 1-[(imidazolidin-2-yl)imino]indazole, combinations thereof, analogs thereof, as well as pharmaceutically acceptable salts thereof (e.g., brimonidine tartrate). As long as a particular isomer, salt, analog, prodrug or other derivative of a selective α-2 adrenergic receptor agonist functions as a selective α-2 agonist, it may be used for the purposes of the present disclosure.

Other redness reduction agents that may be used, include, but are not limited to, phentolamine, naphazoline, tetrahydrozoline, methoxamine, ephedrine, phenylephrine, other vasoconstrictors, combinations thereof, as well as pharmaceutically acceptable salts thereof (e.g., naphazoline hydrochloride and tetrahydrozoline hydrochloride).

In some embodiments of the ophthalmic compositions of the present disclosure in LDPE container closure systems, the redness reduction agents may be present at a concentration from about 0.0001% (w/v) to about 0.050% (w/v). In some embodiments of the compositions, the redness reduction agents may be present at a concentration from about 0.001% (w/v) to about (w/v). In some embodiments of the compositions, the redness reduction agents may be present at a concentration from about 0.010% (w/v) to about 0.050% (w/v), such as from 0.010% (w/v) to 0.050% (w/v). In some embodiments of the compositions, the redness reduction agents may be present at a concentration from about 0.001% (w/v) to about 0.025% (w/v), such as from (w/v) to 0.025% (w/v). In some embodiments of the compositions, the redness reduction agents may be present at a concentration from about 0.010% (w/v) to about 0.025% (w/v), such as from 0.010% (w/v) to 0.025% (w/v).

It is preferred that a concentration of a redness reduction agent is below its vasoconstriction vs. concentration plateau. Typically, the optimal concentration is 10% to 90% above the minimal threshold of measurable vasoconstriction for a particular agent, or below that of the plateau maximum concentration, and is preferably within the about 25% to about 75% range of either of these benchmarks. The term “plateau maximum concentration” means the concentration above which there is no or minimal further vasoconstriction effect. Other considerations in choosing an agent are blood brain permeability and any possible side effects and other systemic reactions.

In some embodiments of the ophthalmic compositions of the present disclosure in LDPE container closure systems, the redness reduction agent is brimonidine and/or a pharmaceutically acceptable salt thereof, which may be present at concentrations from about (w/v) to about 0.050% (w/v). In some embodiments, brimonidine and/or a pharmaceutically acceptable salt thereof may be present at a concentration from about 0.010% (w/v) to about 0.050% (w/v). In some embodiments, brimonidine and/or a pharmaceutically acceptable salt thereof may be present at a concentration from about 0.015% (w/v) to about 0.045% (w/v), such as from 0.015% (w/v) to 0.045% (w/v). In some embodiments, brimonidine and/or a pharmaceutically acceptable salt thereof may be present at a concentration from about 0.015% (w/v) to about 0.035% (w/v), such as from 0.015% (w/v) to 0.035% (w/v). In some embodiments, brimonidine and/or a pharmaceutically acceptable salt thereof may be present at a concentration from about 0.020% (w/v) to about 0.030% (w/v), such as from 0.020% (w/v) to 0.030% (w/v). In some embodiments, brimonidine and/or a pharmaceutically acceptable salt thereof may be present at a concentration of about 0.020% (w/v), about 0.025% (w/v), about 0.030% (w/v), such as from 0.025% (w/v) to 0.030% (w/v).

The ophthalmic compositions of the present disclosure in LDPE container closure systems may also include additional non-therapeutic components, which include, but are not limited to, preservatives, delivery vehicles, tonicity agents, buffers, pH adjustors, antioxidants, and water.

The ophthalmic compositions of the present disclosure in LDPE container closure systems are adjusted with a tonicity agent. In some embodiments, the ophthalmic composition is adjusted with a tonicity agent to a given osmolality. In some embodiments, the tonicity agent is a nonionic tonicity agent chosen from glycerol, urea, sorbitol, mannitol, propylene glycol, dextrose, and other ophthalmically acceptable nonionic tonicity agents. In some embodiments, the ophthalmic compositions of the present disclosure comprise glycerol, propylene glycol, mannitol, sorbitol, or combinations thereof. In other embodiments, the nonionic tonicity agent is used in combination with an ionic salt tonicity agent, such as sodium chloride, at sufficient concentrations to provide a given osmolality.

In some embodiments, the ophthalmic compositions of the present disclosure in LDPE container closure systems are adjusted with two or more tonicity agents, such as two or more non-ionic tonicity agents. In certain embodiments, the compositions comprise glycerol and mannitol. In certain embodiments, the compositions comprise glycerol and propylene glycol. In certain embodiments, the compositions comprise glycerol and sorbitol. In certain embodiments, the compositions comprise mannitol and propylene glycol. In certain embodiments, the ophthalmic compositions of the present disclosure comprise glycerol, propylene glycol, and mannitol.

When present in the ophthalmic compositions of the present disclosure in LDPE container closure systems, each tonicity agent may be used in a concentration of about 0.1% (w/v) to about 10.0% (w/v). In some embodiments, the compositions have a tonicity agent in concentration of about 0.5% (w/v) to about 3.5% (w/v), such as 0.5% (w/v), 1.0% (w/v), 1.5% (w/v), 2.0% (w/v), 2.5% (w/v), 3.0% (w/v), or 3.5% (w/v). In some embodiments, the composition has glycerol in a concentration of about 0.5% (w/v) to about 6.0% (w/v), such as from about 1.0% (w/v) to about 2.0% (w/v), including 1.0% (w/v), 1.1% (w/v), 1.2% (w/v), 1.3% (w/v), 1.4% (w/v), 1.5% (w/v), 1.6% (w/v), 1.7% (w/v), 1.8% (w/v), 1.9% (w/v), and 2.0% (w/v) In some embodiments, the composition has mannitol in a concentration of about 0.5% (w/v) to about 2.3% (w/v), such as from about 1.0% (w/v) to about 2.0% (w/v), including 2.0% (w/v). In some embodiments, the composition has propylene glycol in a concentration of about 0.5% (w/v) to about 1.5% (w/v), including 1.0% (w/v). In some embodiments, the composition has sorbitol in a concentration of about 1.0% (w/v) to about 2.5% (w/v), including 2.0% (w/v).

In some embodiments, the ophthalmic compositions of the present disclosure in LDPE container closure systems are isotonic. In some embodiments, the compositions are adjusted to approximate the osmotic pressure of normal lachrymal fluids, which, as stated in U.S. Pat. No. 6,274,626, is equivalent to a 0.9% solution of sodium chloride or a 2.5% solution of glycerol. Osmotic pressure, measured as osmolality, is generally about 225 mOsm/kg to about 400 mOsm/kg for conventional ophthalmic compositions.

In some embodiments, the ophthalmic compositions of the present disclosure in LDPE container closure systems have an osmolality of about 225 mOsm/kg to about 400 mOsm/kg, such as 225 mOsm/kg to 400 mOsm/kg. In some embodiments, the compositions have an osmolality of about 275 mOsm/kg to about 385 mOsm/kg, such as of 275 mOsm/kg to 385 mOsm/kg. In some embodiments, the compositions have an osmolality of about 285 mOsm/kg to about 355 mOsm/kg, such as 285 mOsm/kg to 355 mOsm/kg. In some embodiments, the compositions have an osmolality of about 275 mOsm/kg to about 325 mOsm/kg, such as about 275, about 280, about 285, about 290, about 295, about 300, about 305, about 315, about 320, or about 325 mOsm/kg. In some embodiments, the composition has an osmolality of about 290 mOsm/kg. In some embodiments, the composition has an osmolality of about 295 mOsm/kg. In some embodiments, the composition has an osmolality of about 300 mOsm/kg.

However, in further embodiments, the ophthalmic compositions of the present disclosure in LDPE container closure systems may be formulated to osmolality in the range from about 400 to about 875 mOsm/kg, for some desired purposes. In particular, such osmolality may be employed if the composition is formulated to be further well tolerated by a user. For example, US Patent Appl. Publ. No. 2006/0148899, incorporated herein by reference in its entirety, provides for ophthalmic compositions having an osmolality from 400 to 875 mOsm/kg, which have been found to provide comfort to a user.

In some embodiments, the ophthalmic composition of the present disclosure in LDPE container closure systems includes one or more preservatives. For example, a preservative may be used when the composition is packaged for multidose units but may be absent from the composition in single dose units of the composition. The ophthalmic compositions of the present disclosure exhibit preservative efficacy according to USP/EP criteria, including when borate free. In some embodiments, the ophthalmic compositions of the present disclosure exhibit preservative efficacy according to USP/EP criteria at low benzalkonium chloride concentrations, including at 0 ppm BAK, when borate free.

When a preservative is to be present, any preservative for ophthalmic compositions may be used, including, but are not limited to, benzalkonium chloride, chlorobutanol, thimerosal, phenylmercuric acetate, benzethonium chloride, cetrimide, stabilized oxychloro complex, chlorite, or phenylmercuric nitrate. However, the ophthalmic compositions of the present disclosure are borate free. When a preservative is used in the composition, the preservative is typically provided in a concentration of about 0.0005% (w/v) to about 10.0% (w/v), although other concentrations may be used.

In other embodiments, the ophthalmic composition of the present disclosure in LDPE container closure systems are preservative free. Such preservative free compositions can be packaged either in a single-dose unit container closure system or inside a preservative free multidose container closure system. In some embodiments, the preservative free ophthalmic compositions of the present disclosure are packed in a single-dose unit container closure system intended for single-use by the patient. In other embodiments, the preservative free ophthalmic compositions of the present disclosure are packed in preservative free multidose container closure systems that enable the composition to be kept sterile/germ-free even after multiple uses by the patient.

In some embodiments, the ophthalmic composition of the present disclosure in LDPE container closure systems comprises benzalkonium chloride in an effective amount as a preservative. In some embodiments, the composition comprises from about 0.0005% (w/v) to about 10.0% (w/v) benzalkonium chloride. In some embodiments, the composition comprises from about 0.002% (w/v) to about 1.0% (w/v) benzalkonium chloride. In some embodiments, the composition comprises from about 0.002% (w/v) to about 0.50% (w/v) benzalkonium chloride, such as from 0.002% (w/v) to about 0.50% (w/v). In some embodiments, the composition comprises from about 0.002% (w/v) to about 0.050% (w/v) benzalkonium chloride, such as from (w/v) to about 0.050% (w/v). In some embodiments, the composition comprises from about 0.002% (w/v) to about 0.004% (w/v) benzalkonium chloride, such as from 0.002% (w/v) to about 0.004% (w/v).

In some embodiments, the composition comprises about 0.0005% (w/v) benzalkonium chloride. In some embodiments, the composition comprises about 0.001% (w/v) benzalkonium chloride. In some embodiments, the composition comprises about 0.0015% (w/v) benzalkonium chloride. In some embodiments, the composition comprises about 0.002% (w/v) benzalkonium chloride. In some embodiments, the composition comprises about 0.0025% (w/v) benzalkonium chloride. In some embodiments, the composition comprises about 0.003% (w/v) benzalkonium chloride. In some embodiments, the composition comprises about 0.0035% (w/v) benzalkonium chloride. In some embodiments, the composition comprises about 0.004% (w/v) benzalkonium chloride.

In some embodiments, the ophthalmic composition of the present disclosure in LDPE container closure systems has no benzalkonium chloride.

In some embodiments, the ophthalmic composition of the present disclosure in LDPE container closure systems may include one or more acids or bases to adjust the pH of the composition and/or may include a buffer to achieve (and maintain) the desired pH of the compositions.

In some embodiments, the ophthalmic composition of the present disclosure in LDPE container closure systems includes one or more pH adjustors. It is understood that acids or bases can be used to adjust the pH of the composition as needed. When a pH adjustor is to be present, any pH adjustor for ophthalmic compositions may be used, including, but are not limited to hydrochloric acid, sodium hydroxide, and tromethamine. Typically, only small amounts of an acid or base will be needed to adjust the pH of the composition, such as 0.05% to 0.1% of a 1N solution.

The pH of the composition is adjusted for optimization of stability and patient tolerability. In some embodiments, the ophthalmic composition of the present disclosure is adjusted to a pH between about 4.4 to about 6.0, although the compositions may also have a pH outside of this range. In some embodiments, the composition has a pH between about 5.2 to about such as between 5.2 to 5.8. In some embodiments, the composition has a pH between about to about 5.75. In some embodiments, the composition has a pH of 4.4, 4.5, 4.6, 4.7, 4.8, 4.8, 5.1, 5.2, 5.3, 5.4, 5.5, 5.6, 5.7, 5.8, 5.9, or 6.0.

In some embodiments, the ophthalmic composition of the present disclosure in LDPE container closure systems includes one or more buffers. When a buffer is to be present, any buffer for ophthalmic compositions may be used, including, but are not limited to, acetate buffers, bicarbonate buffers, citrate buffers, and phosphate buffers, potassium salt buffers, sodium salt buffers. However, the ophthalmic compositions of the present disclosure are borate free. If present, a buffer is used in the ophthalmic composition at a concentration above its “break point concentration,” which is defined generally as the concentration of a buffer that is insufficient to maintain the pH of a solution comprising one or more active agents at a temperature for a given time duration. The buffer is typically provided in a concentration from about 1 millimolar (“mM”) to about 10 mM, such as from about 2 mM to about 6 mM and from about 3 mM to about 4 mM. It has been observed that in some embodiments, buffers decrease the stability of the active ingredients (such as ketotifen). Accordingly, in some embodiments, the ophthalmic composition of the present disclosure is buffer free.

In some embodiments, the ophthalmic composition of the present disclosure in LDPE container closure systems includes one or more antioxidants. In some embodiments, the ophthalmic composition of the present disclosure does not include antioxidants. When an antioxidant is to be present, any antioxidant for ophthalmic compositions may be used, including, but are not limited to, sodium metabisulfite, sodium thiosulfate, acetylcysteine, butylated hydroxyanisole and butylated hydroxytoluene.

In some embodiments, the ophthalmic composition of the present disclosure in LDPE container closure systems includes one or more delivery vehicles. Delivery vehicles include, but are not limited to, polyvinyl alcohol, povidone, hydroxypropyl methyl cellulose, poloxamers, carboxymethyl cellulose, hydroxyethyl cellulose, and water (purified, distilled, deionized). It is also possible to use a physiological saline solution as a major vehicle.

In some embodiments, the ophthalmic composition of the present disclosure in LDPE container closure systems further comprises an effective amount of an additional therapeutic agent, such as an additional active ingredient.

In some embodiments, the additional therapeutic agent is chosen from antihistamines. In some embodiments, the additional therapeutic agent is chosen from pheniramine maleate, carbinoxamine maleate, S-(+)-chlorpheniramine maleate, diphenhydramine, hydroxyzine, and azelastine. In some embodiments, the additional therapeutic agent is pheniramine maleate. In some embodiments, the additional therapeutic agent is chosen from antihistamines, anti-inflammatory agents, and antibiotics. In some embodiments, the additional therapeutic agent is chosen from anti-inflammatory agents and antibiotics. In some embodiments, the additional therapeutic agent is chosen from anti-inflammatory agents. In some embodiments, the additional therapeutic agent is chosen from antibiotics. In some embodiments, the additional therapeutic agent is chosen from fluoroquinolones. In some embodiments, the additional therapeutic agent is besifloxacin.

In some embodiments, the ophthalmic composition of the present disclosure in LDPE container closure systems further comprises a pharmaceutically acceptable excipient chosen from humectants, suspending agents, viscosity-increasing agents, wetting agents, solubilizing agents, chelating agents, stabilizers, and nitrous oxide inhibitors.

In some embodiments, the suspending agents and viscosity-increasing agents are chosen from povidone K90, USP/EP, HPMC E15LV (USP), polycarbophil, HPMC E4M USP, and combinations thereof.

In some embodiments, the wetting agents and solubilizing agents are chosen from povidone, poloxamer 407, PS80, and combinations thereof. In some embodiments, the ophthalmic composition of the present disclosure includes povidone. When present in the ophthalmic compositions of the present disclosure, povidone may additionally act as a lubricant, protecting against irritation and dryness.

When present in the ophthalmic composition as a wetting agent, povidone is used at a concentration from about 0.010% (w/v) to about 1.20% (w/v). In some embodiments, povidone may be present at a concentration from about 0.050% (w/v) to about 1.00% (w/v). In some embodiments, povidone may be present at a concentration from about 0.075% (w/v) to about (w/v), such as from 0.075% (w/v) to about 0.75% (w/v). In some embodiments, povidone may be present at a concentration from about 0.15% (w/v) to about 0.45% (w/v), such as from (w/v) to about 0.45% (w/v). In some embodiments, povidone may be present at a concentration of about 0.15% (w/v), about 0.20% (w/v), about 0.25% (w/v), about 0.30% (w/v), about 0.35% (w/v), about 0.40% (w/v), or about 0.45% (w/v). In some embodiments, the ophthalmic compositions of the present disclosure, which comprise povidone, do not cause a stinging sensation and are comfortable to the eye when applied. Povidone has been observed to improve the stability of the anti-allergen (such as ketotifen fumarate) of the ophthalmic compositions of the present disclosure under high temperature stress conditions.

In some embodiments, the chelating agent is chosen from disodium EDTA, dihydrate USP/EP, and combinations thereof.

In some embodiments, the stabilizers are chosen from ascorbic acid, citric acid, sodium benzoate, calcium propionate, sodium erythorbate, sodium nitrite, calcium sorbate, potassium sorbate, BHA, BHT, EDTA, tocopherols, and combinations thereof. In other embodiments, the stabilizer contains an anionic component, such as peroxide class preservative. The stabilizer allows one to achieve greater penetration of lipophilic membranes. However, in some embodiments, the ophthalmic composition of the present disclosure does not include an antioxidant.

In some embodiments, the nitrous oxide inhibitors are chosen from L-NAME (L-NG-Nitroarginine methyl ester), L-NIL (N-6-(1-iminoethyl)-L-lysine dihydrochloride), L-NIO (N-5-(1-iminoethyl)-L-ornithine dihydrochloride), L-canavine, and combinations thereof.

In some embodiments, the ophthalmic composition comprises (a) an anti-allergen and (b) a redness reduction agent, wherein the composition is in an LDPE container closure system.

In some embodiments, the ophthalmic composition comprises (a) an anti-allergen, (b) a redness reduction agent at a concentration from about 0.010% (w/v) to about 0.050% (w/v), (c) two or more non-ionic tonicity agents in an amount such that the composition has an osmolality of about 275 mOsm/kg to about 385 mOsm/kg, (d) povidone at a concentration from about 0.15% (w/v) to about 0.45% (w/v), (e) an optional preservative, and (f) water, wherein the composition is in an LDPE container closure system.

In some embodiments, the ophthalmic composition comprises (a) an anti-allergen and (b) brimonidine and/or a pharmaceutically acceptable salt thereof, wherein the composition is in an LDPE container closure system.

In some embodiments, the ophthalmic composition comprises (a) an anti-allergen, (b) brimonidine and/or a pharmaceutically acceptable salt thereof at a concentration from about 0.010% (w/v) to about 0.050% (w/v), (c) two or more non-ionic tonicity agents in an amount such that the composition has an osmolality of about 275 mOsm/kg to about 385 mOsm/kg, (d) povidone at a concentration from about 0.15% (w/v) to about 0.45% (w/v), (e) an optional preservative, and (f) water, wherein the composition is in an LDPE container closure system.

In some embodiments, the ophthalmic composition comprises (a) ketotifen or a pharmaceutically acceptable salt thereof and (b) brimonidine or a pharmaceutically acceptable salt thereof, wherein the composition is in an LDPE container closure system.

In some embodiments, the ophthalmic composition comprises (a) ketotifen and/or a pharmaceutically acceptable salt thereof at a concentration from about 0.010% (w/v) to about (w/v), (b) brimonidine and/or a pharmaceutically acceptable salt thereof at a concentration from about 0.010% (w/v) to about 0.050% (w/v), (c) two or more non-ionic tonicity agents in an amount such that the composition has an osmolality of about 275 mOsm/kg to about 385 mOsm/kg, (d) povidone at a concentration from about 0.15% (w/v) to about 0.45% (w/v), (e) an optional preservative, and (f) water, wherein the composition is in an LDPE container closure system.

In some embodiments, the ophthalmic composition comprises (a) ketotifen fumarate and (b) brimonidine tartrate, wherein the composition is in an LDPE container closure system.

In some embodiments, the ophthalmic composition comprises (a) ketotifen fumarate at a concentration from about 0.010% (w/v) to about 0.050% (w/v), (b) brimonidine tartrate from about 0.010% (w/v) to about 0.050% (w/v), (c) two or more non-ionic tonicity agents in an amount such that the composition has an osmolality of about 275 mOsm/kg to about 385 mOsm/kg, (d) povidone at a concentration from about 0.15% (w/v) to about 0.45% (w/v), (e) an optional preservative, and (f) water, wherein the composition is in an LDPE container closure system.

In some embodiments, the ophthalmic composition comprises (a) olopatadine or a pharmaceutically acceptable salt thereof and (b) brimonidine or a pharmaceutically acceptable salt thereof, wherein the composition is in an LDPE container closure system.

In some embodiments, the ophthalmic composition comprises (a) olopatadine and/or a pharmaceutically acceptable salt thereof at a concentration from about 0.100% (w/v) to about (w/v), (b) brimonidine and/or a pharmaceutically acceptable salt thereof at a concentration from about 0.010% (w/v) to about 0.050% (w/v), (c) two or more non-ionic tonicity agents in an amount such that the composition has an osmolality of about 275 mOsm/kg to about 385 mOsm/kg, (d) povidone at a concentration from about 0.15% (w/v) to about 0.45% (w/v), (e) an optional preservative, and (f) water, wherein the composition is in an LDPE container closure system.

In some embodiments, the ophthalmic composition comprises (a) olopatadine HCl and (b) brimonidine tartrate, wherein the composition is in an LDPE container closure system.

In some embodiments, the ophthalmic composition comprises (a) olopatadine HCl at a concentration from about 0.100% (w/v) to about 0.250% (w/v), (b) brimonidine tartrate from about (w/v) to about 0.050% (w/v), (c) two or more non-ionic tonicity agents in an amount such that the composition has an osmolality of about 275 mOsm/kg to about 385 mOsm/kg, (d) povidone at a concentration from about 0.15% (w/v) to about 0.45% (w/v), (e) an optional preservative, and (f) water, wherein the composition is in an LDPE container closure system.

The present disclosure also provides a container closure system containing an ophthalmic composition comprising (a) an anti-allergen and (b) a redness reduction agent, wherein the container closure system is in an LDPE container closure system.

In some embodiments, the container closure system contains an ophthalmic composition comprising (a) an anti-allergen, (b) a redness reduction agent at a concentration from about 0.010% (w/v) to about 0.050% (w/v), (c) two or more non-ionic tonicity agents in an amount such that the composition has an osmolality of about 275 mOsm/kg to about 385 mOsm/kg, (d) povidone at a concentration from about 0.15% (w/v) to about 0.45% (w/v), (e) an optional preservative, and (f) water, wherein the container closure system is in an LDPE container closure system.

In some embodiments, the container closure system contains an ophthalmic composition comprising (a) an anti-allergen and (b) brimonidine and/or a pharmaceutically acceptable salt thereof, wherein the container closure system is in an LDPE container closure system.

In some embodiments, the container closure system contains an ophthalmic composition comprising (a) an anti-allergen, (b) brimonidine and/or a pharmaceutically acceptable salt thereof at a concentration from about 0.010% (w/v) to about 0.050% (w/v), (c) two or more non-ionic tonicity agents in an amount such that the composition has an osmolality of about 275 mOsm/kg to about 385 mOsm/kg, (d) povidone at a concentration from about 0.15% (w/v) to about (w/v), (e) an optional preservative, and (f) water, wherein the container closure system is in an LDPE container closure system.

In some embodiments, the container closure system contains an ophthalmic composition comprising (a) ketotifen or a pharmaceutically acceptable salt thereof and (b) brimonidine or a pharmaceutically acceptable salt thereof, wherein the container closure system is in an LDPE container closure system.

In some embodiments, the container closure system contains an ophthalmic composition comprising (a) ketotifen and/or a pharmaceutically acceptable salt thereof at a concentration from about 0.010% (w/v) to about 0.050% (w/v), (b) brimonidine and/or a pharmaceutically acceptable salt thereof at a concentration from about 0.010% (w/v) to about (w/v), (c) two or more non-ionic tonicity agents in an amount such that the composition has an osmolality of about 275 mOsm/kg to about 385 mOsm/kg, (d) povidone at a concentration from about 0.15% (w/v) to about 0.45% (w/v), (e) an optional preservative, and (f) water, wherein the container closure system is in an LDPE container closure system.

In some embodiments, the container closure system contains an ophthalmic composition comprising (a) ketotifen fumarate and (b) brimonidine tartrate, wherein the container closure system is in an LDPE container closure system.

In some embodiments, the container closure system contains an ophthalmic composition comprising (a) ketotifen fumarate at a concentration from about 0.010% (w/v) to about 0.050% (w/v), (b) brimonidine tartrate from about 0.010% (w/v) to about 0.050% (w/v), (c) two or more non-ionic tonicity agents in an amount such that the composition has an osmolality of about 275 mOsm/kg to about 385 mOsm/kg, (d) povidone at a concentration from about 0.15% (w/v) to about 0.45% (w/v), (e) an optional preservative, and (f) water, wherein the container closure system is in an LDPE container closure system.

In some embodiments, the container closure system contains an ophthalmic composition comprising (a) olopatadine or a pharmaceutically acceptable salt thereof and (b) brimonidine or a pharmaceutically acceptable salt thereof, wherein the container closure system is in an LDPE container closure system.

In some embodiments, the container closure system contains an ophthalmic composition comprising (a) olopatadine and/or a pharmaceutically acceptable salt thereof at a concentration from about 0.100% (w/v) to about 0.250% (w/v), (b) brimonidine and/or a pharmaceutically acceptable salt thereof at a concentration from about 0.010% (w/v) to about (w/v), (c) two or more non-ionic tonicity agents in an amount such that the composition has an osmolality of about 275 mOsm/kg to about 385 mOsm/kg, (d) povidone at a concentration from about 0.15% (w/v) to about 0.45% (w/v), (e) an optional preservative, and (f) water, wherein the container closure system is in an LDPE container closure system.

In some embodiments, the container closure system contains an ophthalmic composition comprising (a) olopatadine HCl and (b) brimonidine tartrate, wherein the composition is in an LDPE container closure system.

In some embodiments, the container closure system contains an ophthalmic composition comprising (a) olopatadine HCl at a concentration from about 0.100% (w/v) to about 0.250% (w/v), (b) brimonidine tartrate from about 0.010% (w/v) to about 0.050% (w/v), (c) two or more non-ionic tonicity agents in an amount such that the composition has an osmolality of about 275 mOsm/kg to about 385 mOsm/kg, (d) povidone at a concentration from about 0.15% (w/v) to about 0.45% (w/v), (e) an optional preservative, and (f) water, wherein the container closure system is in an LDPE container closure system.

The present disclosure also provides a kit comprising a container closure system containing an ophthalmic composition comprising (a) an anti-allergen and (b) a redness reduction agent, wherein the container closure system is in an LDPE container closure system. In some embodiments, kits can optionally further include a second container comprising a suitable aqueous carrier for dilution or suspension of the provided ophthalmic composition for preparation of administration to a subject. In some embodiments, contents of provided formulation container and solvent container combine to form at least one unit dosage form.

In one embodiment, a single container can comprise one or more compartments for containing a provided ophthalmic composition, and/or appropriate aqueous carrier for suspension or dilution. In some embodiments, a single container can be appropriate for modification such that the container can receive a physical modification so as to allow combination of compartments and/or components of individual compartments. For example, a foil or plastic bag can comprise two or more compartments separated by a perforated seal which can be broken so as to allow combination of contents of two individual compartments once the signal to break the seal is generated. A kit can thus comprise such multi-compartment containers including the ophthalmic composition and an appropriate solvent and/or an appropriate aqueous carrier for suspension.

In some embodiments, the kit comprising a container closure system contains an ophthalmic composition comprising (a) an anti-allergen, (b) a redness reduction agent at a concentration from about 0.010% (w/v) to about 0.050% (w/v), (c) two or more non-ionic tonicity agents in an amount such that the composition has an osmolality of about 275 mOsm/kg to about 385 mOsm/kg, (d) povidone at a concentration from about 0.15% (w/v) to about 0.45% (w/v), (e) an optional preservative, and (f) water, wherein the container closure system is in an LDPE container closure system.

In some embodiments, the kit comprising a container closure system contains an ophthalmic composition comprising (a) an anti-allergen and (b) brimonidine and/or a pharmaceutically acceptable salt thereof, wherein the container closure system is in an LDPE container closure system.

In some embodiments, the kit comprising a container closure system contains an ophthalmic composition comprising (a) an anti-allergen, (b) brimonidine and/or a pharmaceutically acceptable salt thereof at a concentration from about 0.010% (w/v) to about (w/v), (c) two or more non-ionic tonicity agents in an amount such that the composition has an osmolality of about 275 mOsm/kg to about 385 mOsm/kg, (d) povidone at a concentration from about 0.15% (w/v) to about 0.45% (w/v), (e) an optional preservative, and (f) water, wherein the container closure system is in an LDPE container closure system.

In some embodiments, the kit comprising a container closure system contains an ophthalmic composition comprising (a) ketotifen or a pharmaceutically acceptable salt thereof and (b) brimonidine or a pharmaceutically acceptable salt thereof, wherein the container closure system is in an LDPE container closure system.

In some embodiments, the kit comprising a container closure system contains an ophthalmic composition comprising (a) ketotifen and/or a pharmaceutically acceptable salt thereof at a concentration from about 0.010% (w/v) to about 0.050% (w/v), (b) brimonidine and/or a pharmaceutically acceptable salt thereof at a concentration from about 0.010% (w/v) to about (w/v), (c) two or more non-ionic tonicity agents in an amount such that the composition has an osmolality of about 275 mOsm/kg to about 385 mOsm/kg, (d) povidone at a concentration from about 0.15% (w/v) to about 0.45% (w/v), (e) an optional preservative, and (f) water, wherein the container closure system is in an LDPE container closure system.

In some embodiments, the kit comprising a container closure system contains an ophthalmic composition comprising (a) ketotifen fumarate and (b) brimonidine tartrate, wherein the container closure system is in an LDPE container closure system.

In some embodiments, the kit comprising a container closure system contains an ophthalmic composition comprising (a) ketotifen fumarate at a concentration from about 0.010% (w/v) to about 0.050% (w/v), (b) brimonidine tartrate from about 0.010% (w/v) to about 0.050% (w/v), (c) two or more non-ionic tonicity agents in an amount such that the composition has an osmolality of about 275 mOsm/kg to about 385 mOsm/kg, (d) povidone at a concentration from about 0.15% (w/v) to about 0.45% (w/v), (e) an optional preservative, and (f) water, wherein the container closure system is in an LDPE container closure system.

In some embodiments, the kit comprising a container closure system contains an ophthalmic composition comprising (a) olopatadine or a pharmaceutically acceptable salt thereof and (b) brimonidine or a pharmaceutically acceptable salt thereof, wherein the container closure system is in an LDPE container closure system.

In some embodiments, the kit comprising a container closure system contains an ophthalmic composition comprising (a) olopatadine and/or a pharmaceutically acceptable salt thereof at a concentration from about 0.100% (w/v) to about 0.250% (w/v), (b) brimonidine and/or a pharmaceutically acceptable salt thereof at a concentration from about 0.010% (w/v) to about 0.050% (w/v), (c) two or more non-ionic tonicity agents in an amount such that the composition has an osmolality of about 275 mOsm/kg to about 385 mOsm/kg, (d) povidone at a concentration from about 0.15% (w/v) to about 0.45% (w/v), (e) an optional preservative, and (f) water, wherein the container closure system is in an LDPE container closure system.

In some embodiments, the kit comprising a container closure system contains an ophthalmic composition comprising (a) olopatadine HCl and (b) brimonidine tartrate, wherein the composition is in an LDPE container closure system.

In some embodiments, the kit comprising a container closure system contains an ophthalmic composition comprising (a) olopatadine HCl at a concentration from about 0.100% (w/v) to about 0.250% (w/v), (b) brimonidine tartrate from about 0.010% (w/v) to about 0.050% (w/v), (c) two or more non-ionic tonicity agents in an amount such that the composition has an osmolality of about 275 mOsm/kg to about 385 mOsm/kg, (d) povidone at a concentration from about 0.15% (w/v) to about 0.45% (w/v), (e) an optional preservative, and (f) water, wherein the container closure system is in an LDPE container closure system.

In some embodiments, the ophthalmic composition of the present disclosure is sterile, according to USP/EP criteria. In some embodiments, sterility is conferred by any conventional method. In some embodiments, sterility is conferred by filtration. In some embodiments, sterility is conferred by irradiation. In some embodiments, sterility is conferred by heating. In some embodiments, sterility is conferred by conducting the manufacturing process under aseptic conditions.

The proportion and nature of any pharmaceutically acceptable excipient may be determined by the chosen route of administration and standard pharmaceutical practice. One of ordinary skill in the art can readily select the proper form and route of administration depending upon the disorder or condition to be treated, the stage of the disorder or condition, and other relevant circumstances.

In some embodiments, the ophthalmic composition can be administered by any convenient route. In some embodiments, the ophthalmic composition is in any liquid form suitable for topical application. In some embodiments, the ophthalmic composition is topically administered to the ocular surface. In some embodiments, the ophthalmic composition is topically administered to the cornea. In some embodiments, the ophthalmic composition is instilled into the conjunctival sac.

In some embodiments the composition is the form of an eye drop, a suspension, a gel, an ointment, an injectable solution, or a spray. In some embodiments, the ophthalmic composition is the form of an eye drop. In some embodiments, the ophthalmic composition is the form of a suspension. In some embodiments, the ophthalmic composition is the form of a gel. In some embodiments, the ophthalmic composition is the form of an ointment. In some embodiments, the ophthalmic composition is the form of an injectable solution. In some embodiments, the ophthalmic composition is the form of a spray. In some embodiments, the ophthalmic composition is in the form of artificial tears. In some embodiments, the ophthalmic composition is in the form of a contact lens adsorbent comprising a liquid carrier, such as, e.g., a cellulose ether (e.g., methylcellulose).

Proper dosages of the ophthalmic compositions of the present disclosure are concentration-dependent. To determine the specific dose for preventing allergic response and whitening of the eyes of a specific person, a skilled artisan would have to take into account kinetics and absorption characteristics of the particular anti-allergen and redness reduction agent. In addition, the dosage may be dependent on the route of administration. The dosages may also de dependent on the degree of allergy relief and/or whitening desired by a patient.

Methods of treating comprise administration of a daily dose of an ophthalmic composition of the present disclosure to a subject in need thereof. Determining and adjusting an appropriate dosing regimen (e.g., adjusting the number of doses and frequency of dosing) per day can be performed by one of ordinary skill in the relevant art, and will depend upon various factors such as the nature and progression of a disorder and/or condition associated with allergies and eye redness, the health, and/or age of the subject. In some embodiments, the ophthalmic composition of the present disclosure is administered to a subject in a single dose per day, in two doses per day, in three doses per day, in four doses per day, etc. or up to, for example, ten doses per day. In some embodiments, the composition is administered to a subject for at least one week, at least two weeks, at least three weeks, at least one month, at least 2 months, at least 3 months, at least 4 months, at least 5 months, at least 6 months, at least 12 months, at least one year, or for more than one year.

Before describing exemplary embodiments of the present disclosure, it is to be understood that the disclosure is not limited to the details of construction or process steps set forth in the following examples and is capable of other embodiments and of being practiced or being carried out in various ways.

### EMBODIMENTS

Without limitation, some embodiments of the disclosure include:

Embodiment 1. An ophthalmic compositions comprising: (a) an anti-allergen and (b) a redness reduction agent.

Embodiment 2. The ophthalmic composition according to Embodiment 1, wherein the redness reduction agent is an adrenergic receptor agonist.

Embodiment 3. The ophthalmic composition according to Embodiment 2, wherein the redness reduction agent is a selective α-2 adrenergic receptor agonist chosen from apraclonidine, mivazerol, clonidine, brimonidine, alpha methyl dopa, guanfacine, dexemeditomidine, (+)-(S)-4-[1-(2,3-dimethyl-phenyl)-ethyl]-1,3-dihydro-imidazole-2-thione, 1-[(imidazolidin-2-yl)imino]indazole, combinations thereof, analogs thereof, and pharmaceutically acceptable salts thereof.

Embodiment 4. The ophthalmic composition according to any one of Embodiments 1 to 3, wherein the redness reduction agent is brimonidine or a pharmaceutically acceptable salt thereof.

Embodiment 5. The ophthalmic composition according to any one of Embodiments 1 to 4, wherein the redness reduction agent is brimonidine tartrate.

Embodiment 6. The ophthalmic composition according to any one of Embodiments 1 to 5, wherein the redness reduction agent is at a concentration from about 0.0001% (w/v) to about (w/v).

Embodiment 7. The ophthalmic composition according to any one of Embodiments 1 to 5, wherein the redness reduction agent is at a concentration from about 0.001% (w/v) to about (w/v).

Embodiment 8. The ophthalmic composition according to any one of Embodiments 1 to 5, wherein the redness reduction agent is at a concentration from about 0.010% (w/v) to about (w/v).

Embodiment 9. The ophthalmic composition according to Embodiment 4 or Embodiment 5, wherein the redness reduction agent is brimonidine or a pharmaceutically acceptable salt thereof present at a concentration from about 0.015% (w/v) to about 0.045% (w/v).

Embodiment 10. The ophthalmic composition according to Embodiment 4 or Embodiment 5, wherein the redness reduction agent is present at a concentration from about (w/v) to about 0.035% (w/v).

Embodiment 11. The ophthalmic composition according to Embodiment 4 or Embodiment 5, wherein the redness reduction agent is present at a concentration from about (w/v) to about 0.030% (w/v).

Embodiment 12. The ophthalmic composition according to any one of Embodiments 1 to 11, wherein the anti-allergen is chosen from acrivastine, azelastine, bepotastine besilate, cetirizine, chlorpheniramine, cromolyn sodium, desloratadine, ebastine, emedastine, epinastine, fexofenadine, ketotifen, levocabastine, levocetirizine, lodoxamide, loratadine, mizolastine, nedocrimil, norastemizole, olopatadine, combinations thereof, analogs thereof, and pharmaceutically acceptable salts thereof.

Embodiment 13. The ophthalmic composition according to any one of Embodiments 1 to 12, wherein the anti-allergen is ketotifen or a pharmaceutically acceptable salt thereof.

Embodiment 14. The ophthalmic composition according to any one of Embodiments 1 to 13, wherein the anti-allergen is ketotifen fumarate.

Embodiment 15. The ophthalmic composition according to any one of Embodiments 1 to 12, wherein the anti-allergen is olopatadine or a pharmaceutically acceptable salt thereof.

Embodiment 16. The ophthalmic composition according to any one of Embodiments 1 to 12 and 15, wherein the anti-allergen is olopatadine HCl.

Embodiment 17. The ophthalmic composition according to any one of Embodiments 1 to 16, wherein the anti-allergen is present at a concentration from about 0.0001% (w/v) to about (w/v).

Embodiment 18. The ophthalmic composition according to any one of Embodiments 1 to 16, wherein the anti-allergen is present at a concentration from about 0.0001% (w/v) to about (w/v).

Embodiment 19. The ophthalmic composition according to any one of Embodiments 1 to 16, wherein the anti-allergen is present at a concentration from about 0.001% (w/v) to about (w/v).

Embodiment 20. The ophthalmic composition according to Embodiment 13 or Embodiment 14, wherein the anti-allergen is present at a concentration from about 0.010% (w/v) to about 0.050% (w/v).

Embodiment 21. The ophthalmic composition according to Embodiment 13 or Embodiment 14, wherein the anti-allergen is present at a concentration from about 0.020% (w/v) to about 0.040% (w/v).

Embodiment 22. The ophthalmic composition according to any one of Embodiments 1 to 16, wherein the anti-allergen is present at a concentration from about 0.001% (w/v) to about (w/v).

Embodiment 23. The ophthalmic composition according to any one of Embodiments 1 to 16, wherein the anti-allergen is present at a concentration from about 0.010% (w/v) to about (w/v).

Embodiment 24. The ophthalmic composition according to any one of Embodiments 1 to 16, wherein the anti-allergen is present at a concentration from about 0.100% (w/v) to about (w/v).

Embodiment 25. The ophthalmic composition according to Embodiment 15 or Embodiment 16, wherein the anti-allergen is olopatadine or a pharmaceutically acceptable salt thereof present at a concentration from about 0.100% (w/v) to about 0.250% (w/v).

Embodiment 26. The ophthalmic composition according to Embodiment 15 or Embodiment 16, wherein the anti-allergen is present at a concentration from about 0.100% (w/v) to about 0.150% (w/v).

Embodiment 27. The ophthalmic composition according to any one of claims 1 to 16, further comprising two or more non-ionic tonicity agents.

Embodiment 28. The ophthalmic composition according to Embodiment 27, wherein each non-ionic tonicity agent is chosen from glycerol, urea, sorbitol, mannitol, propylene glycol, and dextrose.

Embodiment 29. The ophthalmic composition according to Embodiment 27, wherein each non-ionic tonicity agent is chosen from glycerol, propylene glycol, mannitol, and sorbitol.

Embodiment 30. The ophthalmic composition according to Embodiment 27, wherein the two or more non-ionic tonicity agents are glycerol and mannitol.

Embodiment 31. The ophthalmic composition according to Embodiment 27, wherein the two or more non-ionic tonicity agents are glycerol and propylene glycol.

Embodiment 32. The ophthalmic composition according to Embodiment 27, wherein the two or more non-ionic tonicity agents are glycerol and sorbitol.

Embodiment 33. The ophthalmic composition according to Embodiment 27, wherein the two or more non-ionic tonicity agents are glycerol, propylene glycol, and mannitol.

Embodiment 34. The ophthalmic composition according to any one of Embodiments 27 to 33, wherein the two or more non-ionic tonicity agents are present in an amount such that the composition has an osmolality of about 275 mOsm/kg to about 385 mOsm/kg.

Embodiment 35. The ophthalmic composition according to any one of Embodiments 1 to 34, further comprising a wetting agent.

Embodiment 36. The ophthalmic composition according to Embodiment 35, wherein the wetting agent is chosen from povidone, poloxamer 407, PS80, and combinations thereof.

Embodiment 37. The ophthalmic composition according to Embodiment 35, wherein the wetting agent is povidone.

Embodiment 38. The ophthalmic composition according to any one of Embodiments 35 to 37, wherein the wetting agent is present at a concentration from about 0.010% (w/v) to about 1.20% (w/v).

Embodiment 39. The ophthalmic composition according to any one of Embodiments to 37, wherein the wetting agent is present at a concentration from about 0.050% (w/v) to about 1.00% (w/v).

Embodiment 40. The ophthalmic composition according to any one of Embodiments to 37, wherein the wetting agent is present at a concentration from about 0.075% (w/v) to about (w/v).

Embodiment 41. The ophthalmic composition according to any one of Embodiments to 37, wherein the wetting agent is present at a concentration from about 0.15% (w/v) to about (w/v).

Embodiment 42. The ophthalmic composition according to any one of Embodiments 1 to 41, further comprising a preservative.

Embodiment 43. The ophthalmic composition according to Embodiment 42, wherein the preservative is chosen from benzalkonium chloride, chlorobutanol, thimerosal, phenylmercuric acetate, benzethonium chloride, cetrimide, stabilized oxychloro complex, chlorite, and phenylmercuric nitrate.

Embodiment 44. The ophthalmic composition according to Embodiment 42, wherein the preservative is benzalkonium chloride.

Embodiment 45. The ophthalmic composition according to any one of Embodiments 42 to 44, wherein the preservative is present at a concentration from about 0.010% (w/v) to about 1.20% (w/v).

Embodiment 46. The ophthalmic composition according any one of Embodiments 42 to 44, wherein the preservative is present at a concentration from about 0.002% (w/v) to about (w/v).

Embodiment 47. The ophthalmic composition according any one of Embodiments 42 to 44, wherein the preservative is present at a concentration from about 0.002% (w/v) to about (w/v).

Embodiment 48. The ophthalmic composition according to any one of Embodiments 1 to 47, wherein the composition further comprises a non-therapeutic component chosen from a delivery vehicle, buffer, pH adjustor, antioxidants, water, and combinations thereof.

Embodiment 49. The ophthalmic composition according to any one of Embodiments 1 to 48, having a pH between about 4.4 to about 6.0.

Embodiment 50. The ophthalmic composition according to any one of Embodiments 1 to 48, having a pH between about 5.2 to about 5.8.

Embodiment 51. The ophthalmic composition according to any one of Embodiments 1 to 50, wherein the composition further comprises an additional therapeutic component.

Embodiment 52. The ophthalmic composition according to any one of Embodiments 1 to 51, wherein the composition is buffer free.

Embodiment 53. The ophthalmic composition according to any one of Embodiments 1 to 52, wherein the composition is borate free.

Embodiment 54. The ophthalmic composition according to any one of Embodiments 1 to 53, wherein the composition is the form of an eye drop, a suspension, a gel, an ointment, an injectable solution, or a spray.

Embodiment 55. An ophthalmic composition comprising: (a) an anti-allergen, (b) a redness reduction agent at a concentration from about 0.010% (w/v) to about 0.050% (w/v), (c) two or more non-ionic tonicity agents in an amount such that the composition has an osmolality of about 275 mOsm/kg to about 385 mOsm/kg, (d) povidone at a concentration from about 0.15% (w/v) to about 0.45% (w/v), (e) an optional preservative, and (f) water.

Embodiment 56. An ophthalmic composition comprising: (a) ketotifen or a pharmaceutically acceptable salt thereof and (b) brimonidine or a pharmaceutically acceptable salt thereof.

Embodiment 57. An ophthalmic composition comprising: (a) ketotifen or a pharmaceutically acceptable salt thereof at a concentration from about 0.010% (w/v) to about (w/v), (b) brimonidine or a pharmaceutically acceptable salt thereof at a concentration from about 0.010% (w/v) to about 0.050% (w/v), (c) two or more non-ionic tonicity agents in an amount such that the composition has an osmolality of about 275 mOsm/kg to about 385 mOsm/kg, (d) povidone at a concentration from about 0.15% (w/v) to about 0.45% (w/v), (e) an optional preservative, and (f) water.

Embodiment 58. An ophthalmic composition comprising: (a) ketotifen fumarate and (b) brimonidine tartrate.

Embodiment 59. An ophthalmic composition comprising: (a) ketotifen fumarate at a concentration from about 0.010% (w/v) to about 0.050% (w/v), (b) brimonidine tartrate from about (w/v) to about 0.050% (w/v), (c) two or more non-ionic tonicity agents in an amount such that the composition has an osmolality of about 275 mOsm/kg to about 385 mOsm/kg, (d) povidone at a concentration from about 0.15% (w/v) to about 0.45% (w/v), (e) an optional preservative, and (f) water.

Embodiment 60. An ophthalmic composition comprising: (a) olopatadine or a pharmaceutically acceptable salt thereof and (b) brimonidine or a pharmaceutically acceptable salt thereof.

Embodiment 61. An ophthalmic composition comprising: (a) olopatadine or a pharmaceutically acceptable salt thereof at a concentration from about 0.100% (w/v) to about (w/v), (b) brimonidine or a pharmaceutically acceptable salt thereof at a concentration from about 0.010% (w/v) to about 0.050% (w/v), (c) two or more non-ionic tonicity agents in an amount such that the composition has an osmolality of about 275 mOsm/kg to about 385 mOsm/kg, (d) povidone at a concentration from about 0.15% (w/v) to about 0.45% (w/v), (e) an optional preservative, and (f) water.

Embodiment 62. An ophthalmic composition comprising: (a) olopatadine HCl and (b) brimonidine tartrate.

Embodiment 63. An ophthalmic composition comprising: (a) olopatadine HCl at a concentration from about 0.100% (w/v) to about 0.250% (w/v), (b) brimonidine tartrate from about (w/v) to about 0.050% (w/v), (c) two or more non-ionic tonicity agents in an amount such that the composition has an osmolality of about 275 mOsm/kg to about 385 mOsm/kg, (d) povidone at a concentration from about 0.15% (w/v) to about 0.45% (w/v), (e) an optional preservative, and (f) water.

Embodiment 64. The ophthalmic composition according to any one of Embodiments 1 to 63, wherein the composition is in an container closure system.

Embodiment 65. The ophthalmic composition according to Embodiment 64, wherein the container closure system is in an LDPE container closure system.

Embodiment 66. The ophthalmic composition according to Embodiment 64, wherein the container closure system is in a white LDPE container closure system.

Embodiment 67. The ophthalmic composition according to Embodiment 64, wherein the container closure system is in an natural LDPE container closure system.

Embodiment 68. The ophthalmic composition according to any one of Embodiments 64 to 67, wherein the container closure system is chosen from vials, ampoules, bottles, tubes, syringes, and dispenser packages.

Embodiment 69. A container closure system containing an ophthalmic composition according to any one of Embodiments 1 to 63.

Embodiment 70. The container closure system according to Embodiment 69, wherein the container closure system is in a LDPE container closure system.

Embodiment 71. The container closure system according to Embodiment 69, wherein the container closure system is in a white LDPE container closure system.

Embodiment 72. The container closure system according to Embodiment 69, wherein the container closure system is in a natural LDPE container closure system.

Embodiment 73. A container closure system containing an ophthalmic composition comprising: (a) an anti-allergen and (b) a redness reduction agent.

Embodiment 74. The container closure system according to any one of Embodiment 69 to 73, wherein the container closure system is chosen from vials, ampoules, bottles, tubes, syringes, and dispenser packages.

Embodiment 75. A kit comprising a container closure system containing an ophthalmic composition according to any one of Embodiment 1 to 63.

Embodiment 76. The kit according to Embodiment 75, wherein the container closure system is in a LDPE container closure system.

Embodiment 77. The kit according to Embodiment 75, wherein the container closure system is in a white LDPE container closure system.

Embodiment 78. The kit according to Embodiment 75, wherein the container closure system is in a natural LDPE container closure system.

Embodiment 79. A kit comprising a container closure system containing an ophthalmic comprising: (a) an anti-allergen and (b) a redness reduction agent.

Embodiment 80. The kit according to any one of claims 75-79, wherein the container closure system is chosen from vials, ampoules, bottles, tubes, syringes, and dispenser packages.

### EXAMPLES

The following examples are intended to be illustrative and are not meant in any way to limit the scope of the disclosure.

### Example 1

Sixteen different formulations of the ophthalmic compositions of the present disclosure may be prepared from the ingredients listed in Tables 1 and 2 using sodium hydroxide and/or hydrochloric acid to adjust the pH of the composition to a pH between about 4.0 to about 6.5, such as about 5.6. Each composition can be formulated into one, two, or more dosage units.

TABLE 1

| | 1 | 2 | 3 | 4 | 5 | 6 | 7 | 8 |
|---|---|---|---|---|---|---|---|---|
| AN1 (% w/v) | 0.035 | 0.035 | 0.035 | 0.035 | 0.035 | 0.035 | 0.035 | 0.035 |
| CW1 (% w/v) | 0.025 | 0.025 | 0.025 | 0.025 | 0.025 | 0.025 | 0.025 | 0.025 |
| TA1 (% w/v) | 1.50 | 1.40 | 1.00 | 1.50 | 1.50 | 1.50 | 1.50 | |
| TA2 (% w/v) | | 1.00 | 1.00 | 0.50 | | | | 1.00 |
| TA3 (% w/v) | 2.00 | | | 1.00 | 2.00 | 2.00 | | |
| TA4 (% w/v) | | | | | | | 2.00 | 2.00 |
| WA1 (% w/v) | 0.15 | 0.30 | 0.75 | 0.45 | 0.30 | 0.08 | 0.08 | 0.30 |
| PR1 (% w/v) | 0.003 | | 0.003 | 0.003 | 0.003 | 0.003 | 0.003 | 0.003 |
| Purified | q.s. to | q.s. to | q.s. to | q.s. to | q.s. to | q.s. to | q.s. to | q.s. to |
| Water | 100% | 100% | 100% | 100% | 100% | 100% | 100% | 100% |

TABLE 2

| | 9 | 10 | 11 | 12 | 13 | 14 | 15 | 16 |
|---|---|---|---|---|---|---|---|---|
| AN2 (% w/v) | 0.111 | 0.111 | 0.111 | 0.111 | 0.111 | 0.175 | 0.111 | 0.111 |
| CW1 (% w/v) | 0.025 | 0.025 | 0.025 | 0.025 | 0.025 | 0.025 | 0.025 | 0.025 |
| TA1 (% w/v) | 1.50 | 1.50 | 1.00 | 1.50 | 1.50 | 1.50 | 1.50 | |
| TA2 (% w/v) | | 1.00 | 1.00 | 0.50 | | | | 1.00 |
| TA3 (% w/v) | 2.00 | | | 1.00 | 2.00 | 2.00 | | |
| TA4 (% w/v) | | | | | | | 2.00 | 2.00 |
| WA1 (% w/v) | 0.15 | 0.30 | 0.75 | 0.45 | 0.30 | 0.30 | 0.08 | 0.30 |
| PR1 (% w/v) | 0.003 | 0.003 | 0.003 | 0.003 | 0.003 | 0.003 | 0.003 | 0.003 |
| Purified | q.s. to | q.s. to | q.s. to | q.s. to | q.s. to | q.s. to | q.s. to | q.s. to |
| Water | 100% | 100% | 100% | 100% | 100% | 100% | 100% | 100% |

TABLE 3

| | 17 | 18 | 19 | 20 | 21 | 22 | 23 | 24 |
|---|---|---|---|---|---|---|---|---|
| AN1 (% w/v) | 0.030 | 0.030 | 0.030 | 0.030 | 0.030 | 0.030 | 0.030 | 0.030 |
| CW1 (% w/v) | 0.045 | 0.045 | 0.045 | 0.045 | 0.045 | 0.045 | 0.045 | 0.045 |
| TA1 (% w/v) | 1.50 | 1.50 | 1.00 | 1.50 | 1.50 | 1.50 | 1.50 | |
| TA2 (% w/v) | | 1.00 | 1.00 | 0.50 | | | | 1.00 |
| TA3 (% w/v) | 2.00 | | | 1.00 | 2.00 | 2.00 | | 1.00 |
| TA4 (% w/v) | | | | | | | 2.00 | 2.00 |
| WA1 (% w/v) | 0.15 | 0.30 | 0.75 | 0.45 | 0.30 | 0.08 | 0.08 | 0.30 |
| PR1 (% w/v) | 0.003 | 0.003 | 0.003 | 0.003 | 0.003 | 0.003 | 0.003 | 0.003 |
| Purified | q.s. to | q.s. to | q.s. to | q.s. to | q.s. to | q.s. to | q.s. to | q.s. to |
| Water | 100% | 100% | 100% | 100% | 100% | 100% | 100% | 100% |

### Abbreviations

AN1 anti-allergen (Ketotifen Fumarate)
AN2 anti-allergen (Olopatadine HCL)
CW1 redness reduction agent (Brimonidine Tartrate)
TA1 tonicity agent (Glycerol)
TA2 tonicity agent (Propylene Glycol)
TA3 tonicity agent (Mannitol)
TA4 tonicity agent (Sorbitol)
WA1 wetting agent (Povidone)
PR1 preservative (Benzalkonium Chloride, 25% solution)

### Example 2

The stability of an ophthalmic composition of the present disclosure was studied over a period of 13.1 weeks at different conditions in two low density polyethylene (LDPE) container closure systems (white and natural bottles). Samples of the ophthalmic composition of formulation 5 were prepared having a pH of about 5.6 and an osmolality of about 296 mOsm/kg. At 1, 3, 5, 8, and 13.1 weeks, the following were measured: % target (i.e., starting concentration) of the active ingredients, pH, and osmolality. The samples were buffer and borate free. After approximately 3 months (13.1 weeks), the ophthalmic compositions in both container closure systems maintained a potency≥95% for each active ingredient.

TABLE 4
white LDPE bottles (10 mL)

| | | | |
|---|---|---|---|
| Brimonidine | Ketotifen | | |
| tartrate | fumarate | | Osmolality |
| (% target) | (% target) | pH | (mOsm/kg) |

25° C., 60% RH

| | | | | |
|---|---|---|---|---|
| 1 | 102.8 | 100.3 | 5.5 | 299 |
| 3 | 99.8 | 99.5 | 5.6 | 299 |
| 5 | 100.1 | 99.2 | 5.5 | 297 |
| 8 | 99.0 | 98.4 | 5.4 | — |
| 13.1 | 104.8 | 101.5 | 5.5 | 299 |

30° C., 75% RH

| | | | | |
|---|---|---|---|---|
| 1 | 100.6 | 100.0 | 5.5 | 295 |
| 3 | 99.2 | 98.4 | 5.6 | 299 |
| 5 | 100.3 | 99.4 | 5.6 | 298 |
| 8 | 98.9 | 98.1 | 5.4 | — |
| 13.1 | 104.8 | 102.8 | 5.5 | 306 |

40° C., 75% RH

| | | | | |
|---|---|---|---|---|
| 1 | 101.8 | 99.1 | 5.5 | 298 |
| 3 | 100.7 | 98.5 | 5.5 | 299 |
| 5 | 98.9 | 97.3 | 5.5 | 299 |
| 8 | 99.9 | 98.2 | 5.5 | — |
| 13.1 | 100.0 | 97.1 | 5.4 | 300 |

50° C., RH uncontrolled

| | | | | |
|---|---|---|---|---|
| 1 | 103.7 | 101.0 | 5.5 | 300 |
| 3 | 100.9 | 98.4 | 5.4 | 303 |
| 5 | 100.2 | 98.7 | 5.4 | 307 |
| 8 | 100.1 | 95.5 | 5.6 | — |
| 13.1 | 106.9 | 97.0 | 5.2 | 313 |

TABLE 5
natural LDPE bottles (10 mL)

| | | | |
|---|---|---|---|
| Brimonidine | Ketotifen | | |
| tartrate | fumarate | | Osmolality |
| (% target) | (% target) | pH | (mOsm/kg) |

25° C., 60% RH

| | | | | |
|---|---|---|---|---|
| 1 | 101.5 | 99.7 | 5.4 | — |
| 3 | 97.1 | 96.4 | 5.6 | 298 |
| 5 | 100.0 | 99.1 | 5.4 | 297 |
| 8 | 98.3 | 97.3 | 5.0 | — |
| 13.1 | 104.3 | 99.5 | 5.4 | 297 |

30° C., 75% RH

| | | | | |
|---|---|---|---|---|
| 1 | 101.7 | 100.0 | 5.4 | — |
| 3 | 102.8 | 101.5 | 5.5 | 297 |
| 5 | 101.1 | 99.8 | 5.4 | 297 |
| 8 | 101.2 | 99.7 | 5.3 | — |
| 13.1 | 103.6 | 101.3 | 5.4 | 297 |

40° C., 75% RH

| | | | | |
|---|---|---|---|---|
| 1 | 102.8 | 100.0 | 5.4 | — |
| 3 | 101.7 | 98.7 | 5.4 | 297 |
| 5 | 100.1 | 97.8 | 5.3 | 298 |
| 8 | 101.3 | 98.7 | 5.3 | — |
| 13.1 | 104.7 | 98.7 | 5.3 | 298 |

50° C., RH uncontrolled

| | | | | |
|---|---|---|---|---|
| 1 | 102.6 | 98.5 | 5.3 | — |
| 3 | 100.8 | 97.3 | 5.3 | 302 |
| 5 | 102.4 | 98.2 | 5.1 | 303 |
| 8 | 104.4 | 98.5 | 5.5 | — |
| 13.1 | 107.4 | 98.4 | 4.8 | 315 |

### Example 3: Influence of Povidone on Stability

To assess whether povidone affected stability of the ophthalmic compositions of the present disclosure, formulation 1 was prepared four times with the following differences. Formulation 1A has no povidone and the pH was adjusted to about 5.6 with tromethamine and hydrochloric acid. Formulation 1B has 0.15% w/v povidone and the pH was adjusted to about 5.6 with tromethamine and hydrochloric acid. Formulation 1C has no povidone and the pH was adjusted to about 5.6 with sodium hydroxide and hydrochloric acid. Formulation 1D has 0.15% w/v povidone and the pH was adjusted to about 5.6 with sodium hydroxide and hydrochloric acid. The formulations were buffer and borate free.

The stability of an ophthalmic compositions was studied over a period of 18 and sometimes 36 hours at 80° C. in low density polyethylene (LDPE) container closure systems (white and natural 10 mL bottles). Specifically, % target (i.e., starting concentration) of the active ingredients was determined. The ophthalmic compositions in both container closure systems maintained a potency≥95% for each active ingredient. The povidone was not observed to affect the stability of the ophthalmic compositions.

TABLE 6

| | | |
|---|---|---|
| Brimonidine tartrate | Ketotifen fumarate | |
| (% target) | (% target) | LDPE Bottle |

Formulation 1A

| | | | |
|---|---|---|---|
| 18 | 99.0 | 98.0 | white |
| 36 | — | — | white |
| 18 | 99.0 | 97.0 | natural |
| 36 | — | — | natural |

Formulation 1B

| | | | |
|---|---|---|---|
| 18 | 99.0 | 99.0 | white |
| 36 | — | — | white |
| 18 | 99.0 | 98.0 | natural |
| 36 | — | — | natural |

Formulation 1C

| | | | |
|---|---|---|---|
| 18 | 100.0 | 98.0 | white |
| 36 | — | — | white |
| 18 | 101.0 | 99.0 | natural |
| 36 | — | — | natural |

Formulation 1D

| | | | |
|---|---|---|---|
| 18 | 99.0 | 98.0 | white |
| 36 | 98.0 | 96.0 | white |
| 18 | 99.0 | 97.0 | natural |
| 36 | 101.0 | 97.0 | natural |

### Example 4

An ophthalmic composition was prepared from the ingredients listed in Table 7 using sodium hydroxide and/or hydrochloric acid to adjust the pH of the composition to a pH of about 6.5 with an osmolality of about 310 mOsm/kg:

TABLE 7

| Formulation | 25 |
|---|---|
| Ketotifen Fumarate (% w/v) | 0.035 |
| Brimonidine Tartrate (% w/v) | 0.025 |
| Glycerol (% w/v) | 0.996 |
| Boric Acid (% w/v) | 0.916 |
| Sodium Citrate Dihydrate | 0.100 |
| (% w/v) | |
| Sodium Chloride (% w/v) | 0.075 |
| Sodium Borate (% w/v) | 0.054 |
| Potassium Chloride (% w/v) | 0.030 |
| Benzalkonium Chloride, | 0.003 |
| 25% solution (% w/v) | |
| Purified Water | q.s. to 100% |

The stability of the formulation was studied over a period of up to 78 days at different conditions in a glass container closure system and two low density polyethylene (LDPE) container closure systems (white and natural bottles). At different daily intervals, the % target (i.e., starting concentration) of the active ingredients were measured. While the Brimonidine Tartrate maintained a potency≥95% in each container closure system, the Ketotifen Fumarate did so only when in a glass bottle.

TABLE 8
glass bottles

| | |
|---|---|
| Brimonidine tartrate | Ketotifen fumarate |
| (% target) | (% target) |

30° C.

| | | |
|---|---|---|
| 0 | 100 | 101 |
| 17 | 100 | 101 |
| 30 | 100 | 100 |
| 78 | 101 | 100 |

40° C.

| | | |
|---|---|---|
| 0 | 100 | 101 |
| 17 | 100 | 102 |
| 30 | 100 | 100 |
| 78 | 103 | 100 |

50° C.

| | | |
|---|---|---|
| 0 | 100 | 101 |
| 17 | 100 | 102 |
| 30 | 100 | 100 |
| 78 | 104 | 96 |

TABLE 9
natural LDPE bottles

| | |
|---|---|
| Brimonidine tartrate | Ketotifen fumarate |
| (% target) | (% target) |

30° C.

| | | |
|---|---|---|
| 0 | 100 | 101 |
| 3 | 100 | 100 |
| 16 | 100 | 97 |
| 64 | 101 | 98 |

40° C.

| | | |
|---|---|---|
| 0 | 100 | 101 |
| 3 | 100 | 98 |
| 16 | 100 | 91 |
| 64 | 103 | 82 |

50° C.

| | | |
|---|---|---|
| 0 | 100 | 101 |
| 3 | 100 | 92 |
| 16 | 100 | 81 |
| 64 | 104 | 61 |

TABLE 10
white LDPE bottles

| | |
|---|---|
| Brimonidine tartrate | Ketotifen fumarate |
| (% target) | (% target) |

30° C.

| | | |
|---|---|---|
| 0 | 100 | 101 |
| 17 | 100 | 87 |
| 30 | 99 | 88 |
| 78 | 102 | 71 |

40° C.

| | | |
|---|---|---|
| 0 | 100 | 101 |
| 17 | 100 | 63 |
| 30 | 100 | 58 |
| 78 | 102 | 40 |

50° C.

| | | |
|---|---|---|
| 0 | 100 | 101 |
| 17 | 101 | 48 |
| 30 | 101 | 34 |
| 78 | 106 | 18 |

### Example 5: Efficacy Test for BAK-Preserved Solutions

Preservation efficacy of the ophthalmic compositions of the present disclosure was determined by evaluating the antimicrobial efficacy dose response of benzalkonium chloride (BAK) after 2 days, 7 days, 14 days, and 28 days. Samples of formulation 1 were prepared with the pH was adjusted to about 5.6 with sodium hydroxide and hydrochloric acid. Log (reduction) values are reported.

TABLE 11

| | | | 0.000 | 0.001 | 0.002 | 0.0025 | 0.003 | 0.004 |
|---|---|---|---|---|---|---|---|---|
| BAK Dose | | | % w/v | % w/v | % w/v | % w/v | % w/v | % w/v |
| Staphylococcus | 2 | Days | 4.7 | >4.7 | >4.7 | >4.7 | >4.7 | >4.7 |
| aureus | 7 | Days | >4.7 | >4.7 | >4.7 | >4.7 | >4.7 | >4.7 |
| | 14 | Days | >4.7 | >4.7 | >4.7 | >4.7 | >4.7 | >4.7 |
| | 28 | Days | >4.7 | >4.7 | >4.7 | >4.7 | >4.7 | >4.7 |
| Pseudomonas | 2 | Days | 2.6 | >4.6 | >4.6 | >4.6 | >4.6 | >4.6 |
| aeruginosa | 7 | Days | >4.6 | >4.6 | >4.6 | >4.6 | >4.6 | >4.6 |
| | 14 | Days | >4.6 | >4.6 | >4.6 | >4.6 | >4.6 | >4.6 |
| | 28 | Days | >4.6 | >4.6 | >4.6 | >4.6 | >4.6 | >4.6 |
| Escherichia coli | 2 | Days | 2.9 | >4.7 | >4.7 | >4.7 | >4.7 | >4.7 |
| | 7 | Days | >4.7 | >4.7 | >4.7 | >4.7 | >4.7 | >4.7 |
| | 14 | Days | >4.7 | >4.7 | >4.7 | >4.7 | >4.7 | >4.7 |
| | 28 | Days | >4.7 | >4.7 | >4.7 | >4.7 | >4.7 | >4.7 |
| Candida albicans | 2 | Days | 0.1 | 1.9 | 3.3 | 3.3 | 3.7 | 4.1 |
| | 7 | Days | 0.8 | 4.6 | 4.6 | 4.6 | 4.6 | 4.6 |
| | 14 | Days | 2.6 | >4.6 | >4.6 | >4.6 | >4.6 | >4.6 |
| | 28 | Days | 4.2 | >4.6 | >4.6 | >4.6 | >4.6 | >4.6 |
| Aspergillus | 2 | Days | −0.5 | 0.6 | 0.8 | 0.9 | 0.9 | 1.0 |
| brasiliensis | 7 | Days | −0.2 | 0.5 | 1.1 | 1.1 | 1.2 | 1.5 |
| | 14 | Days | 0 | 1.9 | 1.2 | 1.4 | 1.3 | 1.6 |
| | 28 | Days | 0.4 | 1.1 | 1.4 | 1.5 | 1.8 | 2.1 |
| USP Criteria* | | | Meets | Meets | Meets | Meets | Meets | Meets |

*USP Category IA Criteria:
(1) The concentration of viable bacteria show not less than 1.0 log reduction from the initial calculated count at 7 days. not less than 3.0 log reduction from the initial count of 14 days. and no increase from the 14 day count at 28 days.
(2) The concentrations of yeasts and molds show no increase from the initial calculated count at 7, 14 and 28 days.
(3) No increase is defined as NMT 0.5 log10 unit higher than the previous value measured.

### Example 6

The stability of an ophthalmic composition of the present disclosure was studied over a period of up to 18 months at different conditions in natural low density polyethylene (LDPE) container closure systems with different sizes and different file sizes. Samples of the ophthalmic composition of formulation 5 were prepared. At 0, 3, 6, 9, 12, and/or 18 months, the following were measured: % target of the active ingredients, pH, osmolality, and total degradation products. The samples were buffer and borate free. After approximately 3, 6, 9, 12, and 18 months, the ophthalmic compositions in both container closure systems maintained a potency≥95% for each active ingredient.

TABLE 12
natural LDPE bottle (4 ml with 2 ml fill)

| | | | | |
|---|---|---|---|---|
| Brimonidine | Ketotifen | | | |
| tartrate | fumarate | | Osmolality | Total Degradation |
| (% target) | (% target) | pH | (mOsm/kg) | Products (%) |

25° C., 40% RH

| | | | | | |
|---|---|---|---|---|---|
| 0 months | 99.1 | 98.8 | 5.43 | 289 | <0.05 |
| 3 months | — | — | — | — | — |
| 6 months | 99.5 | 98.5 | 5.4 | 296 | 0.07 |
| 9 months | 101.1 | 98.4 | 5.5 | 296 | 0.17 |
| 12 months | 101.8 | 99.2 | 5.4 | 300 | 0.15 |
| 18 months | 103.2 | 100 | 5.4 | 300 | 0.17 |

30° C., 65% RH

| | | | | | |
|---|---|---|---|---|---|
| 0 months | 99.1 | 98.8 | 5.43 | 289 | <0.05 |
| 3 months | — | — | — | — | — |
| 6 months | 98.7 | 97.1 | 5.3 | 295 | 0.07 |
| 9 months | 100.6 | 97.8 | 5.4 | 296 | 0.12 |
| 12 months | 101.3 | 98.0 | 5.3 | 296 | 0.13 |
| 18 months | — | — | — | — | — |

40° C., 20% RH

| | | | | | |
|---|---|---|---|---|---|
| 0 months | 99.1 | 98.8 | 5.43 | 289 | <0.05 |
| 3 months | 102.2 | 97.3 | 5.3 | 300 | 0.22 |
| 6 months | 105.4 | 97.9 | 5.3 | 317 | 0.70 |
| 9 months | — | — | — | — | — |
| 12 months | — | — | — | — | — |
| 18 months | — | — | — | — | — |

TABLE 13
natural LDPE bottle (10 ml with 2.5 ml fill)

| | | | | |
|---|---|---|---|---|
| Brimonidine | Ketotifen | | | |
| tartrate | fumarate | | Osmolality | Total Degradation |
| (% target) | (% target) | pH | (mOsm/kg) | Products (%) |

25° C., 40% RH

| | | | | | |
|---|---|---|---|---|---|
| 0 months | 99.2 | 98.7 | 5.38 | 290 | <0.05 |
| 3 months | — | — | — | — | — |
| 6 months | 100.0 | 98.4 | 5.5 | 295 | 0.15 |
| 9 months | 101.4 | 98.8 | 5.4 | 297 | 0.22 |
| 12 months | 102.3 | 99.3 | 5.3 | 303 | 0.35 |
| 18 months | 104.600000 | 100.6 | 5.2 | 306 | 0.50 |

30° C., 65% RH

| | | | | | |
|---|---|---|---|---|---|
| 0 months | 99.2 | 98.7 | 5.38 | 290 | <0.05 |
| 3 months | — | — | — | — | — |
| 6 months | 99.4 | 97.2 | 5.3 | 298 | 0.24 |
| 9 months | 100.9 | 97.1 | 5.3 | 297 | 0.49 |
| 12 months | 101.7 | 97.7 | 5.3 | 299 | 0.60 |
| 18 months | — | — | — | — | — |

40° C., 20% RH

| | | | | | |
|---|---|---|---|---|---|
| 0 months | 99.2 | 98.7 | 5.38 | 290 | <0.05 |
| 3 months | 103.4 | 97.5 | 5.3 | 304 | 0.49 |
| 6 months | 108.7 | 100.5 | 5.2 | 324 | 1.06 |
| 9 months | — | — | — | — | — |
| 12 months | — | — | — | — | — |
| 18 months | — | — | — | — | — |

TABLE 14
natural LDPE bottle (10 ml with 5 ml fill)

| | | | | |
|---|---|---|---|---|
| Brimonidine | Ketotifen | | | |
| tartrate | fumarate | | Osmolality | Total Degradation |
| (% target) | (% target) | pH | (mOsm/kg) | Products (%) |

25° C., 40% RH

| | | | | | |
|---|---|---|---|---|---|
| 0 months | 98.5 | 98.6 | 5.67 | 291 | <0.05 |
| 3 months | 98.5 | 98.0 | 5.4 | 288 | 0.15 |
| 6 months | 98.5 | 97.7 | 5.4 | 294 | 0.16 |
| 9 months | — | — | — | — | — |
| 12 months | 100.7 | 98.9 | 5.5 | 296 | 0.19 |
| 18 months | 101.8 | 99.4 | 5.3 | 294 | 0.23 |

30° C., 65% RH

| | | | | | |
|---|---|---|---|---|---|
| 0 months | 98.5 | 98.6 | 5.67 | 291 | <0.05 |
| 3 months | — | — | — | — | — |
| 6 months | 98.4 | 96.9 | 5.3 | 294 | 0.19 |
| 9 months | 100.1 | 97.1 | 5.4 | 295 | 0.24 |
| 12 months | 99.7 | 96.1 | 5.3 | 296 | 0.37 |
| 18 months | — | — | — | — | — |

40° C., 20% RH

| | | | | | |
|---|---|---|---|---|---|
| 0 months | 98.5 | 98.6 | 5.67 | 291 | <0.05 |
| 3 months | 101 | 96.7 | 5.4 | 299 | 0.23 |
| 6 months | 103.1 | 97.4 | 5.3 | 309 | 0.56 |
| 9 months | — | — | — | — | — |
| 12 months | — | — | — | — | — |
| 18 months | — | — | — | — | — |

TABLE 15
natural LDPE bottle (10 ml with 7.5 ml fill)

| | | | | |
|---|---|---|---|---|
| Brimonidine | Ketotifen | | | |
| tartrate | fumarate | | Osmolality | Total Degradation |
| (% target) | (% target) | pH | (mOsm/kg) | Products (%) |

25° C., 40% RH

| | | | | | |
|---|---|---|---|---|---|
| 0 months | 98.3 | 98.4 | 5.43 | 289 | <0.05 |
| 3 months | — | — | — | — | — |
| 6 months | 98.5 | 98.2 | 5.4 | 291 | 0.17 |
| 9 months | 99.1 | 97.4 | 5.4 | 296 | 0.18 |
| 12 months | 99.6 | 97.8 | 5.4 | 292 | 0.20 |
| 18 months | 100.6 | 98.4 | 5.4 | 293 | 0.23 |

30° C., 65% RH

| | | | | | |
|---|---|---|---|---|---|
| 0 months | 98.3 | 98.4 | 5.43 | 289 | <0.05 |
| 3 months | — | — | — | — | — |
| 6 months | 98.0 | 97.0 | 5.4 | 295 | 0.20 |
| 9 months | 99.4 | 97.3 | 5.4 | 292 | 0.20 |
| 12 months | 99.1 | 96.8 | 5.4 | 293 | 0.28 |
| 18 months | — | — | — | — | — |

40° C., 20% RH

| | | | | | |
|---|---|---|---|---|---|
| 0 months | 98.3 | 98.4 | 5.43 | 289 | <0.05 |
| 3 months | 99.8 | 96.6 | 5.3 | 296 | 0.24 |
| 6 months | 100.9 | 95.7 | 5.3 | 301 | 0.47 |
| 9 months | — | — | — | — | — |
| 12 months | — | — | — | — | — |
| 18 months | — | — | — | — | — |

What is claimed is:

1. An ophthalmic composition comprising:
(a) ketotifen or a pharmaceutically acceptable salt thereof at a concentration from about 0.010% (w/v) to about 0.050% (w/v),
(b) brimonidine or a pharmaceutically acceptable salt thereof at a concentration from about (w/v) to about 0.050% (w/v),
(c) two or more non-ionic tonicity agents in an amount such that the composition has an osmolality of about 275 mOsm/kg to about 385 mOsm/kg,
(d) povidone at a concentration from about 0.15% (w/v) to about 0.45% (w/v),
(e) an optional preservative, and
(f) water.

2. The ophthalmic composition according to claim 1, wherein each non-ionic tonicity agent is chosen from glycerol, urea, sorbitol, mannitol, propylene glycol, and dextrose.

3. The ophthalmic composition according to claim 1, wherein the preservative is present in the ophthalmic composition and is chosen from benzalkonium chloride, chlorobutanol, thimerosal, phenylmercuric acetate, benzethonium chloride, cetrimide, stabilized oxychloro complex, chlorite, and phenylmercuric nitrate.

4. The ophthalmic composition according to claim 3, wherein the preservative is present at a concentration from about 0.010% (w/v) to about 1.20% (w/v).

5. The ophthalmic composition according to claim 3, wherein the preservative is present at a concentration from about 0.002% (w/v) to about 0.004% (w/v).

6. The ophthalmic composition according to claim 1, wherein the composition is preservative free.

7. The ophthalmic composition according to claim 1, wherein the composition further comprises a non-therapeutic component chosen from a delivery vehicle, buffer, pH adjustor, antioxidants, water, and combinations thereof.

8. The ophthalmic composition according to claim 1, having a pH between about 4.4 to about 6.0.

9. The ophthalmic composition according to claim 1, wherein the composition further comprises an additional therapeutic component.

10. The ophthalmic composition according to claim 1, wherein the composition is buffer free.

11. The ophthalmic composition according to claim 1, wherein the composition is borate free.

12. The ophthalmic composition according to claim 1, wherein the composition is the form of an eye drop, a suspension, a gel, an ointment, an injectable solution, or a spray.

13. The ophthalmic composition of claim 1, wherein
(a) the ketotifen or a pharmaceutically acceptable salt thereof is ketotifen fumarate and
(b) the brimonidine or a pharmaceutically acceptable salt thereof is brimonidine tartrate.

14. The ophthalmic composition of claim 1, wherein each of the ketotifen or a pharmaceutically acceptable salt thereof and the brimonidine or a pharmaceutically acceptable salt thereof of the ophthalmic composition is stable at 25° C. and 40% relative humidity for at least two years, wherein stability is observed when less than 20% of each of the ketotifen or a pharmaceutically acceptable salt thereof and the brimonidine or a pharmaceutically acceptable salt thereof has degraded or changed.

15. The ophthalmic composition of claim 1, wherein each of the ketotifen or a pharmaceutically acceptable salt thereof and the brimonidine or a pharmaceutically acceptable salt thereof of the ophthalmic composition is stable at 25° C. and 40% relative humidity for at least two years, wherein stability is observed when less than 10% of each of the ketotifen or a pharmaceutically acceptable salt thereof and the brimonidine or a pharmaceutically acceptable salt thereof has degraded or changed.

16. An ophthalmic composition comprising:
(a) ketotifen fumarate at a concentration of about 0.035% (w/v),
(b) brimonidine tartrate at a concentration of about 0.025% (w/v),
(c) glycerol at a concentration of about 1.5% (w/v),
(d) mannitol at a concentration of about 2.0% (w/v),
(e) povidone at a concentration of about 0.3% (w/v),
(f) benzalkonium chloride at a concentration of about 0.003% (w/v), and
(g) water,
wherein the ophthalmic composition has a pH between about 4.4 to about 6.0 and an osmolality of about 275 mOsm/kg to about 385 mOsm/kg.

17. The ophthalmic composition of claim 16, wherein each of the ketotifen fumarate and the brimonidine tartrate of the ophthalmic composition is stable at 25° C. and 40% relative humidity for at least two years, wherein stability is observed when less than 20% of each of the ketotifen fumarate and the brimonidine tartrate has degraded or changed.

18. An ophthalmic composition comprising:
(a) olopatadine or a pharmaceutically acceptable salt thereof at a concentration from about (w/v) to about 0.250% (w/v),
(b) brimonidine or a pharmaceutically acceptable salt thereof at a concentration from about (w/v) to about 0.050% (w/v),
(c) two or more non-ionic tonicity agents in an amount such that the composition has an osmolality of about 275 mOsm/kg to about 385 mOsm/kg,
(d) povidone at a concentration from about 0.15% (w/v) to about 0.45% (w/v),
(e) an optional preservative, and
(f) water.

19. The ophthalmic composition of claim 18, wherein
(a) the olopatadine or a pharmaceutically acceptable salt thereof is olopatadine HCl and
(b) the brimonidine or a pharmaceutically acceptable salt thereof is brimonidine tartrate.

20. The ophthalmic composition according to claim 18, wherein the composition is borate free.
